SEQUENCE LISTING (77 sequences)

SEQ ID NO: 1 - DNA, 24 bp, Artificial Sequence
  ST (split trigger)
gagtggatggtgaaggtgaaggta

SEQ ID NO: 2 - DNA, 39 bp, Artificial Sequence
  HP1, showing exemplary placement of the detection moiety
  misc_feature (21)..(22): iCy5 is between residues 21 and 22
gttggaattgggagtaagggctgtgatgcccttactccc

SEQ ID NO: 3 - DNA, 39 bp, Artificial Sequence
  HP2, showing exemplary placement of the detection moiety
  misc_feature (39)..(39): Cy3 is tagged to residue 39
gcccttactcccaattccaacgggagtaagggcatcaca

SEQ ID NO: 4 - DNA, 21 bp, Artificial Sequence
  F (Fluorophore), showing exemplary placement of the detection moiety
  misc_feature (21)..(21): 3-FAM is tagged to residue 21
gttggaattgggagtaagggc

SEQ ID NO: 5 - DNA, 12 bp, Artificial Sequence
  Q (Quencher), showing exemplary placement of the detection moiety
  misc_feature (1)..(1): 5IABkFQ is tagged to residue 1
gcccttactccc

SEQ ID NO: 6 - DNA, 51 bp, Artificial Sequence
  I1-HP1
taccttcacctttttttttttttttttgtgcggcattcaattccaactgcc

SEQ ID NO: 7 - DNA, 46 bp, Artificial Sequence
  I2-HP1
gcccttactcctgccgcactttttttttttttttcaccatccactc

SEQ ID NO: 8 - DNA, 50 bp, Artificial Sequence
  I1-HP2
taccttcacctttttttttttttttttgtgcggcttcaattccaacagcc

SEQ ID NO: 9 - DNA, 45 bp, Artificial Sequence
  I2-HP2
gcccttactccgccgcactttttttttttttttcaccatccactc

SEQ ID NO: 10 - DNA, 49 bp, Artificial Sequence
  I1-HP3 (A4-6)
taccttcacctttttttttttttttttgtgcccttcaattccaacaagg

SEQ ID NO: 11 - DNA, 44 bp, Artificial Sequence
  I2-HP3 (A4-6)
gcccttactccgggcactttttttttttttttcaccatccactc

SEQ ID NO: 12 - DNA, 51 bp, Artificial Sequence
  I1-HP4
taccttcacctttttttttttttttttgtgcgccttcaattccaacaaggc

SEQ ID NO: 13 - DNA, 45 bp, Artificial Sequence
  I2-HP4
gcccttactccggcgcactttttttttttttttcaccatccactc

SEQ ID NO: 14 - DNA, 52 bp, Artificial Sequence
  I1-HP5
taccttcacctttttttttttttttttgtgcaggcttcaattccaacaagcc

SEQ ID NO: 15 - DNA, 46 bp, Artificial Sequence
  I2-HP5 and I2-HP6
gcccttactccgcctgcactttttttttttttttcaccatccactc

SEQ ID NO: 16 - DNA, 53 bp, Artificial Sequence
  I1-HP6
taccttcacctttttttttttttttttgtgcaggcttcaattccaacgaagcc

SEQ ID NO: 17 - DNA, 53 bp, Artificial Sequence
  I1-HP7 (A5-8)
taccttcacctttttttttttttttttgtgcagtgttcaattccaacgaacac

SEQ ID NO: 18 - DNA, 46 bp, Artificial Sequence
  I2-HP7 (A5-8) and I2-HP8
gcccttactcccactgcactttttttttttttttcaccatccactc

SEQ ID NO: 19 - DNA, 54 bp, Artificial Sequence
  I1-HP8
taccttcacctttttttttttttttttgtgcagtgttcaattccaactgaacac

SEQ ID NO: 20 - DNA, 54 bp, Artificial Sequence
  I1-HP9 (A6-9)
taccttcacctttttttttttttttttagtagtggtttcaattccaacgaaacc

SEQ ID NO: 21 - DNA, 47 bp, Artificial Sequence
  I2-HP9 (A6-9)
gcccttactccaccactacttttttttttttttttcaccatccactc

SEQ ID NO: 22 - DNA, 43 bp, Artificial Sequence
  I1-A4
taccttcacctttttttttttttttttgtgcttcaattccaac

SEQ ID NO: 23 - DNA, 42 bp, Artificial Sequence
  I2-A4
gcccttactccgcactttttttttttttttcaccatccactc

SEQ ID NO: 24 - DNA, 44 bp, Artificial Sequence
  I1-A5
taccttcacctttttttttttttttttgtgcattcaattccaac

SEQ ID NO: 25 - DNA, 43 bp, Artificial Sequence
  I2-A5
gcccttactcctgcactttttttttttttttcaccatccactc

SEQ ID NO: 26 - DNA, 45 bp, Artificial Sequence
  I1-A6
taccttcacctttttttttttttttttagtagtttcaattccaac

SEQ ID NO: 27 - DNA, 44 bp, Artificial Sequence
  I2-A6
gcccttactccactacttttttttttttttttcaccatccactc

SEQ ID NO: 28 - DNA, 51 bp, Artificial Sequence
  PM-I1 and I1-HP9 (A6-9) (for single nucleotide mismatch detection)
gaggagatgtttttttttttttttagtagtggtttcaattccaacgaaacc

SEQ ID NO: 29 - DNA, 47 bp, Artificial Sequence
  PM-I2 and I2-HP9 (A6-9) (for single nucleotide mismatch detection)
gcccttactccaccactacttttttttttttttttgttggacagccg

SEQ ID NO: 30 - DNA, 43 bp, Artificial Sequence
  miR-21 (HP-I1)
tcaacatcagtttttttgtgcagtgttcaattccaacgaacac

SEQ ID NO: 31 - DNA, 37 bp, Artificial Sequence
  miR-21 (I2)
gcccttactcccactgcactttttttctgataagcta

SEQ ID NO: 32 - DNA, 42 bp, Artificial Sequence
  miR-let-7a (HP-I1)
aactatacaactttttttttttttttgtgcttcaattccaac

SEQ ID NO: 33 - DNA, 40 bp, Artificial Sequence
  miR-let-7a (I2)
gcccttactccgcacttttttttttttttttactacctca

SEQ ID NO: 34 - DNA, 48 bp, Artificial Sequence
  miR-9 (HP-I1)
tcatacagctatttttttttttttttgtgcccttcaattccaacaagg

SEQ ID NO: 35 - DNA, 44 bp, Artificial Sequence
  miR-9 (I2)
gcccttactccgggcactttttttttttttttgataaccaaaga

SEQ ID NO: 36 - DNA, 46 bp, Artificial Sequence
  miR-29 (HP-I1)
cggaacaccatttttttttttttttgtgcccttcaattccaacaag

SEQ ID NO: 37 - DNA, 35 bp, Artificial Sequence
  miR-29 (I2)
gcccttactccgggcacttttttaaagaaatcagt

SEQ ID NO: 38 - DNA, 52 bp, Artificial Sequence
  SARS-CoV-2 (HP-I1)
tgcaccccgcattactttttttttttttttgtgcccttcaattccaacaagg

SEQ ID NO: 39 - DNA, 47 bp, Artificial Sequence
  SARS-CoV-2 (I2)
gcccttactccgggcactttttttttttttttgtttggtggaccctc

SEQ ID NO: 40 - DNA, 70 bp, Artificial Sequence
  Thrombin (HP-I1)
agtccgtggtagggcaggttggggtgacttttttttttttttttgtgcagtgttcaattccaacgaacac

SEQ ID NO: 41 - DNA, 49 bp, Artificial Sequence
  Thrombin (I2)
gcccttactcccactgcactttttttttttttttggttggtgtggttgg

SEQ ID NO: 42 - DNA, 52 bp, Artificial Sequence
  I1-HP3 (A4-6) (for protein detection)
  misc_feature (1)..(1): SH is tagged to residue 1. The thiolated end “SH-” was later conjugated to antibody
ttttttttttttttttttttttttttttttgtgcccttcaattccaacaagg

SEQ ID NO: 43 - DNA, 47 bp, Artificial Sequence
  I2-HP3 (A4-6) (for protein detection)
  misc_feature (47)..(47): SH is tagged to residue 47. The thiolated end “SH-” was later conjugated to antibody
gcccttactccgggcactttttttttttttttttttttttttttttt

SEQ ID NO: 44 - DNA, 41 bp, Artificial Sequence
  I1-HP7 (A5-8) (for protein detection)
  misc_feature (1)..(1): SH is tagged to residue 1. The thiolated end “SH-” was later conjugated to antibody
tttttttttttttttgtgcagtgttcaattccaacgaacac

SEQ ID NO: 45 - DNA, 34 bp, Artificial Sequence
  I2-HP7 (A5-8) (for protein detection)
  misc_feature (34)..(34): SH is tagged to residue 34. The thiolated end “SH-” was later conjugated to antibody
gcccttactcccactgcacttttttttttttttt

SEQ ID NO: 46 - DNA, 59 bp, Artificial Sequence
  HP-I1 (7)
cctcatccctactttttttttttttttgctagacgaatttcaattccaactgaaattcg

SEQ ID NO: 47 - DNA, 49 bp, Artificial Sequence
  I2 (7)
gcccttactccattcgtctagctttttttttttttttatccatctccac

SEQ ID NO: 48 - DNA, 58 bp, Artificial Sequence
  HP-I1 (8) and HP-I1 (9)
taccttcacctttttttttttttttgctagacgaatttcaattccaacttgaaattcg

SEQ ID NO: 49 - DNA, 51 bp, Artificial Sequence
  I2 (8)
gcccttactccattcgtctagcaatttttttttttttttcaccatccactc

SEQ ID NO: 50 - DNA, 49 bp, Artificial Sequence
  I2 (9)
gcccttactccattcgtctagctttttttttttttttcaccatccactc

SEQ ID NO: 51 - DNA, 60 bp, Artificial Sequence
  HP-I1 (10)
taccttcacctttttttttttttttgctagacgaattttcaattccaacttgaaaattcg

SEQ ID NO: 52 - DNA, 50 bp, Artificial Sequence
  I2 (10)
gcccttactccaattcgtctagctttttttttttttttcaccatccactc

SEQ ID NO: 53 - DNA, 60 bp, Artificial Sequence; identical to SEQ ID NO: 51
  HP-I1 (11)

SEQ ID NO: 54 - DNA, 50 bp, Artificial Sequence; identical to SEQ ID NO: 52
  I2 (11)

SEQ ID NO: 55 - DNA, 60 bp, Artificial Sequence
  HP-I1 (12)
taccttcacctttttttttttttttgctattcgaattttcaattccaacttgaaaattcg

SEQ ID NO: 56 - DNA, 50 bp, Artificial Sequence
  I2 (12)
gcccttactccaattcgaatagctttttttttttttttcaccatccactc

SEQ ID NO: 57 - DNA, 41 bp, Artificial Sequence
  HP-I1 (13)
taccttcaccttttttttacccgattcaattccaacaatcg

SEQ ID NO: 58 - DNA, 35 bp, Artificial Sequence
  I2 (13)
gcccttactcctcgggtttttttcaccatccactc

SEQ ID NO: 59 - DNA, 24 bp, Artificial Sequence
  Exemplary target sequence for Single Nucleotide Mismatch Detection (perfectly matched)
cggctgtccaaccacatctcctcc

SEQ ID NO: 60 - DNA, 24 bp, Artificial Sequence
  Exemplary target sequence for Single Nucleotide Mismatch Detection (single mismatched)
cggctgtccaaccacatcttctcc

SEQ ID NO: 61 - RNA, 22 nt, Artificial Sequence
  miR-21
uagcuuaucagacugauguuga

SEQ ID NO: 62 - RNA, 22 nt, Artificial Sequence
  miR-let-7a
ugagguaguagguuguauaguu

SEQ ID NO: 63 - RNA, 23 nt, Artificial Sequence
  miR-9
ucuuugguuaucuagcuguauga

SEQ ID NO: 64 - RNA, 22 nt, Artificial Sequence
  miR-29
acugauuucuuuugguguucag

SEQ ID NO: 65 - DNA, 72 bp, Artificial Sequence
  SARS-CoV-2 (NC_045512.2)
tctggttactgccagttgaatctgagggtccaccaaacgtaatgcggggtgcatttcgctgattttggggtc

SEQ ID NO: 66 - DNA, 41 bp, Artificial Sequence; identical to SEQ ID NO: 44
  HP-I1 (with biotin-tag)
  misc_feature (1)..(1): Biotin tag is tagged to residue 1

SEQ ID NO: 67 - DNA, 34 bp, Artificial Sequence; identical to SEQ ID NO: 45
  I2 (with biotin-tag)
  misc_feature (34)..(34): Biotin tag is tagged to residue 34

SEQ ID NO: 68 - DNA, 17 bp, Artificial Sequence
  Duplex-cI2
gtgcccggagtaagggc

SEQ ID NO: 69 - DNA, 48 bp, Artificial Sequence
  SM-I1
taccttcacctttttttttttttttttgtgcccttaattccaacaagg

SEQ ID NO: 70 - DNA, 28 bp, Artificial Sequence
  Xprobe-F
  misc_feature (28)..(28): 3Rox_N is tagged to residue 28
gttaaatcgtggatagtagacttcgcac

SEQ ID NO: 71 - DNA, 28 bp, Artificial Sequence
  Xprobe-Q
  misc_feature (1)..(1): 5IAbRQ is tagged to residue 1
gtgcgaacaggtacatttgctcgtcctt

SEQ ID NO: 72 - DNA, 32 bp, Artificial Sequence
  XF-P
aaggacgagcaaatgtacctggcccttactcc

SEQ ID NO: 73 - DNA, 42 bp, Artificial Sequence
  XQ-PC
gttggaattgggagtaagggcgtctactatccacgatttaac

SEQ ID NO: 74 - DNA, 39 bp, Artificial Sequence; identical to SEQ ID NO: 2
  HP1 (AuNP)

SEQ ID NO: 75 - DNA, 39 bp, Artificial Sequence; identical to SEQ ID NO: 3
  HP2 (AuNP)

SEQ ID NO: 76 - DNA, 60 bp, Artificial Sequence
  HP1 (DNAzyme)
gttgggaaagttggaattgggagtaagggctgtgatgcccttactccctgggtagggcgg

SEQ ID NO: 77 - DNA, 48 bp, Artificial Sequence
  HP2 (DNAzyme)
gttgggaaagcccttactcccaattccaacgggagtaagggcatcaca

(19) United States
(12) Patent Application Publication
(10) Pub. No.: US 2022/0389486 A1
(43) Pub. Date: Dec. 8, 2022

(54) PROBE ASSAY FOR THE DETECTION OF BIOMOLECULES

(71) Applicant: NATIONAL UNIVERSITY OF SINGAPORE, Singapore (SG)

(72) Inventors: Lin Yue YUNG, Singapore (SG); Yan Shan ANG, Singapore (SG)

(21) Appl. No.: 17/613,564

(22) PCT Filed: May 22, 2020
(86) PCT No.: PCT/SG2020/050305

§ 371 Date: Nov. 23, 2021

(30) Foreign Application Priority Data
May 24, 2019 (SG) 10201904711X

(51) Int. Cl. C12Q 1/6818

(52) U.S. Cl. CPC C12Q 1/6818; C12Q 1/6834

(57) ABSTRACT

Disclosed herein are compositions for detecting the presence of one or more target(s) of interest, wherein the composition comprises a first hairpin initiator molecule and a second initiator molecule. Also disclosed herein are methods of using the same.

### CROSS-REFERENCE TO RELATED APPLICATIONS

This application claims the benefit of priority of SG provisional application no. 10201904711X, filed 24 May 2019, the contents of it being hereby incorporated by reference in its entirety for all purposes.

### FIELD OF THE INVENTION

The present invention relates generally to the field of molecular biology. In particular, the present invention relates to the use of nucleic acid based detection probes.

### BACKGROUND

Detection and identification of molecules in samples using an experimental assay plays a large role in laboratories. Biomolecular interactions, such as cell receptor clustering for signal transduction, host-cell interaction during infections and within protein complexes, for example, are involved in numerous biological processes. Despite the biological significance, a simple molecular toolbox for the direct detection of such multi-target activities is lacking. Conventional analysis methods often require artificial counterparts to fish for the interacting partner, or extensive sample processing to isolate the biomolecules from their original environment, e.g. pull-down assays and gel retardation assays. DNA circuits are a versatile and highly-programmable toolbox which can be used for autonomous sensing of dynamic events, such as biomolecular interactions. However, experimental implementation of in silico circuit designs has been hindered by the problem of circuit leakage. Thus, there is a need for probe-based assays with a reduced rate of circuit leakage for detecting biomolecules.

### SUMMARY

In one aspect, the present disclosure refers to a composition for detecting the presence of one or more target(s) of interest, wherein the composition comprises the following components: a first hairpin initiator molecule according to structure I, wherein the structure comprises domains a, b₁*, b₂, b₂*, e, e*, s and x*; wherein neighbouring domains are connected directly to each other or via a linker; wherein domain x* binds, or is complementary, to the one or more target(s) of interest; wherein domain b₁* forms a hairpin loop; wherein domains b₂* and b₂ are of the same length and are complementary to each other; wherein domains b₂* and b₂ are at least 2 nucleotides in length; wherein domains e and e* are of the same length, are at least 2 nucleotides in length, and are complementary to each other; wherein domain a is at least 3 nucleotides in length; wherein domain s is a spacer; and a second initiator molecule according to structure II, wherein the structure comprises domains a*, c*, e*, s′ and y*; wherein domain y* is complementary, or binds to, the one or more target(s) of interest; wherein domains a and a* are complementary to each other; wherein domain e* of structure II is at least 2 nucleotides in length and binds to domain e of structure I upon binding the target of interest, wherein domain c* is capable of binding to a signal generating molecule/signal generating complex; and wherein domain s′ is a spacer, wherein the length of domains s and s′ are selected to allow domains b₁*, b₂* and c* to be adjacent to each other upon binding.

In another aspect, the present disclosure refers to a method for detecting one or more target(s) of interest in a sample, the method comprising providing a sample thought to comprise the one or more target(s) of interest and detecting the one or more target(s) of interest using the composition as disclosed herein, whereby each composition is specific for one target of interest.

In yet another aspect, the present disclosure refers to a method for detecting the presence of one or more target(s) of interest in a sample, the method comprising adding one or more compositions as disclosed herein to the sample; allowing binding of the one or more compositions to the one or more target(s) of interest thought to be comprised in the sample; measuring one or more signals resulting from the binding of the composition(s) to the target(s) of interest; wherein the generation of one or more signals detects the presence of one or more of the target(s) of interest in the sample.

In a further aspect, the present disclosure refers to a method of identifying a disease, the method comprising adding one or more compositions as disclosed herein to a sample obtained from a subject suspected to have the disease; allowing binding of the one or more compositions to the one or more target(s) of interest; measuring one or more signals resulting from the binding of the composition(s) to the target(s) of interest; and identifying the disease, wherein the presence of one or more signals indicates the presence of one or more of the target(s) of interest in the sample; and wherein the one or more of the target(s) of interest are disease-specific.

In another aspect, the present disclosure refers to a kit comprising the composition as defined herein.

### BRIEF DESCRIPTION OF THE DRAWINGS

The invention will be better understood with reference to the detailed description when considered in conjunction with the non-limiting examples and the accompanying drawings, in which:

FIG. 1 shows a schematic of the concept of the dynamically elongated association toehold, and the interaction partners of each of the domains shown in the figure. Initiators known in the art for convention association toehold design were modified to contain a hairpin lock, referred to as a hairpin initiator (HP-I1) in this figure. The hairpin lock was characterized by an additional domain e and by using part of a previously disclosed domain b* (now denoted as domain b₂*) as a clamp to maintain the hairpin metastability in absence of trigger event. (Step 1) Upon target binding at the two recognition sites (denoted as domain x and y), (step 2) the two initiators (denoted as HP-I1 and I2, or structures I and II respectively) are brought into proximity to stabilize the hybridization at domain a. (Step 3) The hairpin lock is then partially displaced by domain e* on I2 (structure II) followed by (step 4) the dissociation of domain b₂*. The initial state has a shorter association region (domain a) with low leak rate; while the final state, after the opening of the hairpin lock upon target binding, is effectively stabilized by a longer association region (domain a+e) as a result of this elongation mechanism for stronger signal generation.

FIG. 2 shows results of a comparison of Förster resonance energy transfer (FRET) ratio obtained for different configurations of hairpin initiator 1 (HP-I1; also referred to as structure I) designs. The lengths of domains b₂, e and a for each hairpin design are indicated respectively to the left of the graph. The light grey bar refers to no target added (indicating potential background noise or leakage), while the dark grey bar refers to addition of 20 nM target (indicating signal generation). The signal-to-noise ratio (S/N) for each design is presented as a line-and-scatter plot. N.S. not significant; * p<0.05; ** p<0.01; *** p<0.001. The aim is to achieve the lowest (background) noise possible and at the same time achieved the highest signal possible. Thus, the higher the S/N value, the higher the signal is over the noise.

FIG. 3 shows a graph depicting the signal-to-noise ratio (S/N) for different hairpin initiator 1 (HP-I1) designs. A cut-off line was drawn to highlight the two tiers of S/N—high S/N was attained, for example, by HP3, HP7 and HP9 designs, which were shortlisted for further investigation. This graph shows the effect of different hairpin initiator 1 designs on the “signal to noise ratio” (S/N), whereby, as mentioned above, the aim is to achieve the lowest (background) noise possible and at the same time achieved the highest signal possible. Thus, the higher the S/N value, the higher the signal is over the noise.

FIG. 4 shows results of leakage suppression using the dynamically elongated association toehold concept, the general principle of which is exemplified in FIG. 1. (A) shows the evolution of circuit leakage when the respective initiators were used for split proximity circuit (SPC) reaction in absence of the target. The hairpin initiator is shown to suppress the leakage significantly compared to conventional initiator design with an equivalent effective association length. (B) shows the signal-to-noise ratio (S/N) of the hairpin initiator design was higher than the conventional initiator design regardless of the length of association region.

FIG. 5 shows data illustrating the kinetics and thermodynamics using elongated association toehold. (A) The performance of the hairpin initiator design (domain a→a+e) is compared to a previously designed single-stranded initiator design (domain a only) and after elongation (domain a+e). The hairpin initiator design improved the kinetics and thermodynamics of split proximity circuits (SPC) compared to the conventional initiator design, and approached close to the performance of the elongated association domain, for the effective association lengths of (B) 4→6 nucleotides (nt), (C) 5→8 nucleotides and (D) 6→9 nucleotides. All data is shown as mean±S.D. (n=3).

FIG. 6 shows data of the resulting analytical performance of split proximity circuits (SPC). (A) Kinetics of split proximity circuits (SPC) for A6-9 hairpin initiator design when titrated with 0-10 nM of split trigger (ST). (B) The Förster resonance energy transfer (FRET) ratio depended linearly on the split trigger (ST) concentration for A4-6, A5-8 and A6-9 designs. All data are shown as mean±S.D. (n=3). (C) shown an overview of the limits of detection (LOD; shown in nM) determined for each of the association domains for the hairpin initiator designs disclosed in the depicted table. This information shown in (C) is also presented as Table 1.

FIG. 7 shows data illustrating the kinetics of the split proximity circuit (SPC) evaluated under different Mg²⁺ concentrations in 1×PBS (pH 7.4) using HP3 design at 25° C. All data is shown as mean±S.D (n=3). As the Mg²⁺ concentration increased, the signal-to-background (S/B) ratio improved significantly and the best S/B ratio was obtained for 5 mM Mg^{2±}. The final buffer composition used in this experiment is 1×PBS (pH 7.4) and 5 mM MgCl₂, for 20 nM HP-I1, 20 nM 12, 40 nM HP1 and 20 nM HP2.

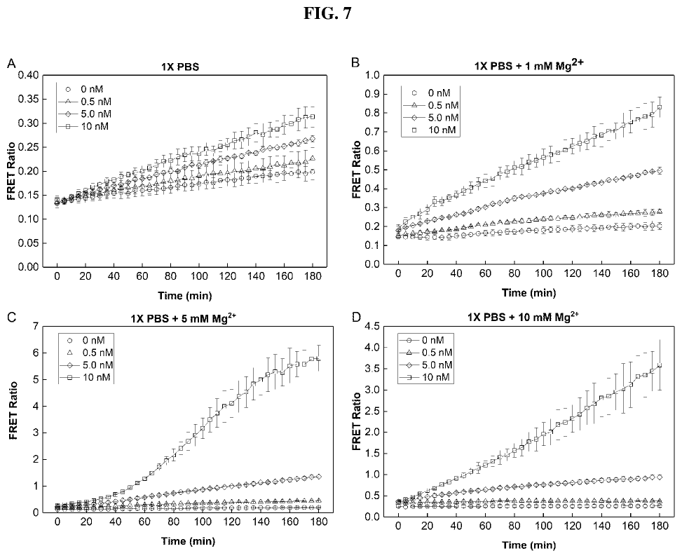

FIG. 8 provides a schematic showing that the DNA-based assay disclosed herein is based on modular design consisting of three key steps of 1) target recognition, 2) signal transduction and 3) signal generation. As one example of the readout signal, hybridization chain reaction was used, indicated by the inclusion of two hairpin monomers (HP1 and HP2), for signal amplification. In this example, four oligonucleotides (or conjugate) probes are mixed and added directly to the sample to produce fluorescence signal (FRET) in the presence of target.

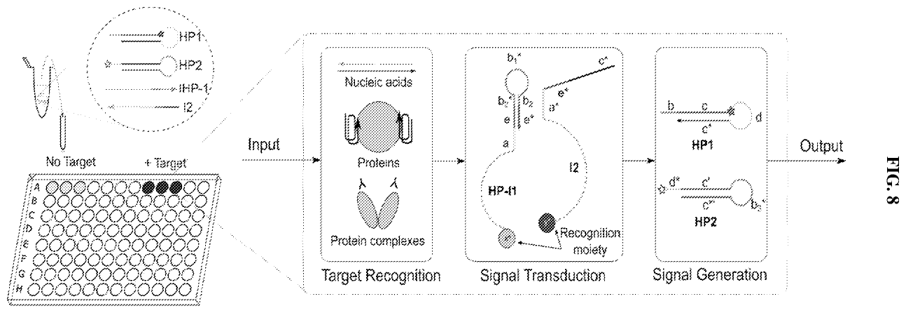

FIG. 9 shows data pertaining to examples of other classes of biomolecules which can be detected using the composition disclosed herein. This indicates the universal application of the subject matter disclosed in the present application. (A) shows a heatmap of the Förster resonance energy transfer (FRET) ratio obtained when different combinations of initiators were reacted with different viral strands separately for 30 minutes. (B) shows the results of the FRET ration obtained in the analysis of seven (7) G6PD models: Canton, Riverside, Union, Mahidol, Mediterranean (Med), A+ and Kaiping. “NC” refers to a negative control set-up, where no target strand was added to the reaction. * p<0.05; ** p<0.01 (one-tailed Student's t-test). (C) shows data indicating that the circuit discriminated between the microRNA of let-7 family members, with high selectivity for let-7a which the probe was designed for. ** refers to p<0.01 (n=3); *** refers to p<0.005 (n=3). (D) shows data indication that the circuit detected HER2:HER3 heterodimers (bright dots), which are cell surface protein receptors present on breast cancer cells (AU565). The image was obtained using confocal microscopy after 30 minutes of incubation. All data are shown as mean±S.D. (n=3) wherever relevant.

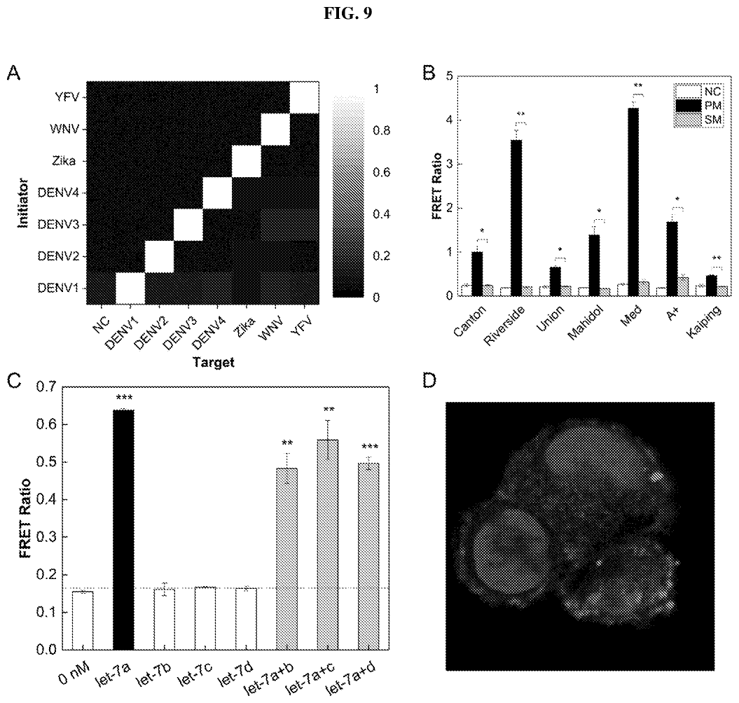

FIG. 10 shows one example of signal generation using the composition disclosed herein. Following on from the last construct as shown in FIG. 1, when HP-I1 and I2 are brought into proximity, a complete trigger strand (represented by domains c* and b*) is now presented. The complete trigger strand is then used to generate readout signal via downstream DNA hybridization reactions. In this example, it triggers the opening of two metastable hairpins (for example, HP1 and HP2) in a cascaded manner to form a long, amplified DNA chain via a process called hybridization chain reaction and generating, in this example, a Förster resonance energy transfer (FRET) signal as the readout signal, indicating presence of and binding to a target of interest.

### DETAILED DESCRIPTION OF THE PRESENT INVENTION

Discussed herein is a molecule that has been designed based on the concept of split proximity circuits (SPCs). A split proximity circuit is a versatile and highly-programmable toolbox, which can be used for the autonomous sensing of target molecules, such as nucleic acids, proteins and protein complexes, or of dynamic events, such as biomolecular interactions. Such circuits can be generated using nucleic acids, such as, but not limited to, DNA and RNA. It was thought to develop split proximity circuits which can boost the association kinetics without incurring additional circuit leakage. As used herein, the term “circuit leakage” refers to the background signal generated by random hybridization between DNA strands in absence of one or more target(s) of interest.

Association toehold is a flexible concept, where the toehold and branch migration domains are decoupled on separate DNA strands which can then be activated later to reassemble in response to specific hybridization events. It builds upon the concept of remote toehold, where toehold-mediated strand displacement which can proceed across non-adjacent domains, albeit with penalized kinetics. The conventional association toehold involves the dynamic reassembly of the complete trigger domains found on separate single-stranded DNA strands upon addition of specific inputs. Typically, the rate limiting step is the hybridization of the short association region. As both the desired signal and undesired leak reactions (causing background noise) can be determined by a single parameter, i.e. the length of association domain, there is an inherent limitation of such systems where a longer association domain necessarily leads to both stronger signal generation and higher background noise. As used herein, the term “toehold” refers to a short single-stranded nucleic acid segment that colocalizes the reactant DNA molecules and initiates a strand displacement reaction. The term “association toehold” refers to one form of toehold design wherein the active circuit domains are located on separate nucleic acid strands and are brought into adjacent positions by an activating event, for example target binding.

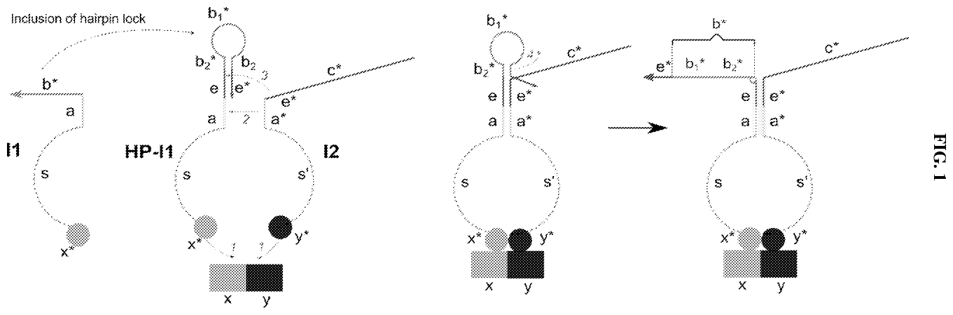

Molecules and compositions based on the concept of a modified association toehold using a previously-reported split proximity circuit (SPC) are disclosed herein. The modified association toehold was characterized by the dynamic elongation of the association region when triggered by specific targets thereby reconciling the trade-offs between the rates of desired reaction and leakage (FIG. 1). By way of an example, this dynamic elongation was achieved by adding a hairpin lock design in initiator 1 (I1), which included an additional domain e (corresponding to the extent of elongation) and part of a previously disclosed toehold domain b* (denoted herein as domain b₂*) as a clamp to maintain the hairpin metastability (see Ang et al., Nucleic Acids Research, 2016, Vol. 44, No. 14 e121). The split proximity circuit (SPC) published in Ang et al. was based on the conventional association toehold design which suffered from the trade-off between increased signal generation and increased circuit leakage when the association length is increased. Hence, the older design reported in Ang et al. (2016), which also pertains to linear initiators as opposed to the hairpin initiators disclosed herein, suffered slow circuit kinetics and signal-to-noise ratio which limited its practical application. The hairpin initiator design introduced in this disclosure enabled a longer association domain to be used, after elongation mediated by the hairpin lock, which sped up the circuit kinetics by more than four-fold (using the design based on the present disclosure) and signal-to-noise ratio by up to 10-fold.

The initial length of the exposed association region in free solution corresponded to that of domain a. Upon target binding, the two initiator strands were brought into close proximity in accordance with the split proximity circuit design. This promoted the opening of the hairpin lock on I1 via binding to domains a and e, which effectively became the new, elongated association region. The domain b₂* clamp dissociated spontaneously to expose remaining toehold domain b₁*. The final assembly retained previously disclosed trigger domains (c* b*), albeit with a domain e* overhang at the 3′ end of I1 conferring improved stability to the ST-I1-I2 assembly due to the longer association region (which is the combined length of domains a and e).

Thus, in one example, there is disclosed a composition for detecting the presence of one or more target(s) of interest. In one example, the composition comprises a first hairpin initiator molecule according to structure I:

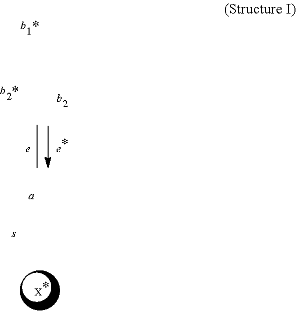

wherein the structure comprises domains a, b₁*, b₂, b₂*, e, e*, s and x*. For clarity's sake, the domains denoted with an asterisk indicate domains which bind to a domain with the same name, but without the asterisk. For example, as shown in FIG. 1, domain a and domain a* bind to each other; domain x binds to domain x* and domain y binds to domain y*. In one example, neighbouring domains are connected directly to each other or via a linker; domain x* binds, or is complementary, to the one or more target(s) of interest; domain b₁* forms a hairpin loop; domains b₂* and b₂ are of the same length and are complementary to each other, domains b₂* and b₂ are at least 2 nucleotides in length; domains e and e* are of the same length and are complementary to each other; domains e and e* are at least 2 nucleotides in length; domain a is at least 3 nucleotides in length; domain s is a spacer of variable length, wherein the spacer length is selected to allow binding of domain x* and a domain y* of a second initiator molecule to the one or more target(s) of interest. In another example, domains b₁*, b₂* and c* are to be adjacent to each other. This is the case, for example, upon target binding. That is to say that target binding serves to enable domains b₁*, b₂* and c* to be adjacent to each other.

Disclosed herein is also a second initiator molecule according to structure II

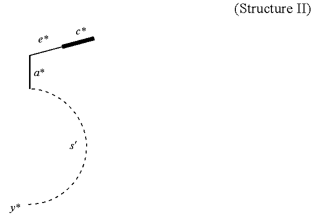

wherein the structure comprises domains a*, c*, e*, s′ and y*. In one example, domain y* is complementary, or binds to, the one or more target(s) of interest adjacent to or downstream of domain x, wherein domain x is found on the target of interest; domains a and a* are complementary to each other; domain e* is as defined herein; domain c* is structured to bind to a signal generating molecule/signal generating complex; and wherein domain s′ is as defined herein.

In another example, the composition comprises the following components: a first hairpin initiator molecule according to structure I, wherein the structure comprises domains a, b₁*, b₂, b₂*, e, e*, s and x*; wherein neighbouring domains are connected directly to each other or via a linker; wherein domain x* binds, or is complementary, to the one or more target(s) of interest; wherein domain b₁* forms a hairpin loop; wherein domains b₂* and b₂ are of the same length and are complementary to each other; wherein domains b₂* and b₂ are at least 2 nucleotides in length; wherein domains e and e* are of the same length and are complementary to each other; wherein domains e and e* are at least 2 nucleotides in length; wherein domain a is at least 4 nucleotides in length; wherein domain s is a spacer of variable length, wherein the spacer length is selected to allow binding of domain x* and a domain y* of a second initiator molecule to the one or more target(s) of interest; and a second initiator molecule according to structure II, wherein the structure comprises domains a*, c*, e*, s and y*; wherein domain y* is complementary, or binds to, the one or more target(s) of interest adjacent to or downstream of domain x, wherein domain x is found on the target of interest; wherein domains a and a* are complementary to each other; wherein domain e* is as defined herein, wherein domain c* is structured to bind to a signal generating molecule/signal generating complex; and wherein domain s is as defined herein.

In yet another example, the composition comprises the following components: a first hairpin initiator molecule according to structure I, wherein the structure comprises domains a, b₁*, b₂, b₂*, e, e*, s and x*; wherein neighbouring domains are connected directly to each other or via a linker; wherein domain x* binds, or is complementary, to the one or more target(s) of interest; wherein domain b₁* forms a hairpin loop; wherein domains b₂* and b₂ are of the same length and are complementary to each other; wherein domains b₂* and b₂ are at least 2 nucleotides in length; wherein domains e and e* are of the same length, are at least 2 nucleotides in length, and are complementary to each other; wherein domain a is at least 3 nucleotides in length; wherein domain s is a spacer; and a second initiator molecule according to structure II, wherein the structure comprises domains a*, c*, e*, s′ and y*; wherein domain y* is complementary, or binds to, the one or more target(s) of interest; wherein domains a and a* are complementary to each other; wherein domain e* of structure II is at least 2 nucleotides in length and binds to domain e of structure I upon binding the target of interest, wherein domain c* is capable of binding to a signal generating molecule/signal generating complex; and wherein domain s′ is a spacer, wherein the length of domains s and s′ are selected to allow domains b₁*, b₂* and c* to be adjacent to each other upon binding.

Such an initiator molecule can comprise secondary structures, for example, but not limited to, stem loops, helixes, duplex structures, hairpin loops and the like. In one example, the initiator molecule can comprise duplex structures and hairpin loops. In another example, the initiator molecule disclosed herein has a linear structure.

Binding of the structure disclosed herein to the one or more targets of interest(s) is dependent on the target recognition domains x* and y* (also termed target binding domains). As appreciated by the person skilled in the art, the binding of structures I and II in close proximity of each other, close the circuit and generate a signal. In this case, the term “proximity” refers to the distance between structure I and II. The term “close proximity” indicates a distance between structures I and II with allow binding to each other. These structures are not considered to be in close proximity to each other in the event that binding of structures I and II to each other and to the target of interest is not given.

It is to be understood that the closing of the circuit, as disclosed herein, that is the binding of the initiator molecules to a target of interest indicates the presence of such a target. Thus, in one example, the presence of the one or more target(s) of interest indicates the presence of a disease. In another example, the presence of the one or more target(s) of interest can indicate the absence of a disease. Also, when used in a quantitative manner, the signals generated by the binding of the initiator molecules to a target of interest can give an indication, or directly translate to, of an increase or decrease in the concentration of a target of interest. This can be compared to, for example, a baseline measurement (for example, when comparing global expression or metabolic changes), or, in another example, could be used to compare concentrations of a target of interest in a disease-free or healthy subject in view of the same target of interest in a diseases subject.

Thus, also disclosed herein is a method of detecting/identifying the presence of a disease. In another example, there is disclosed a of identifying a disease, the method comprising adding one or more compositions as disclosed herein to a sample obtained from a subject suspected to have the disease; allowing binding of the one or more compositions to the one or more target(s) of interest suspected to be comprised in the sample; measuring one or more signals resulting from the binding of the composition(s) to the target(s) of interest; and identifying the disease, wherein the presence of one or more signals indicates the presence of one or more of the target(s) of interest in the sample; and wherein the one or more of the target(s) of interest are disease-specific.

In some examples, target recognition or binding domains x* and y* can be connected to the remaining structure by way of, for example, a spacer sequence s or s′, as disclosed herein. This is the case in situations where the target(s) of interest are so voluminous that binding of the target recognition domains x* and y* would result in, for example, steric hindrance. Or, for example, in some cases, the binding sites of domains x* and y* on the target of interest are so far apart that structures I and II are unable to bind to each other once bound to the target of interest. In such situations, the use of a spacer sequence s can overcome the issues or limitations directly resulting from steric hindrance or distance between structures I and II once bound to the target of interest, giving the structures disclosed herein flexibility to fulfil the dual function of binding to each other and the target of interest. A person skilled in the art would appreciate that the spacer sequences s and s′ are chosen so as to allow for domains b₁*, b₂* and c* to be adjacent to each other, facilitated by the activation via domains a and e. Thus, in one example, the spacer sequences in structure I and II are the same. In another example, the spacer sequences present in structures I and II are different from each other. In one example, spacer sequences s and s′ are symmetrical. In another example, spacer sequences s and s′ are not symmetrical.

In one example, domains s and s′ are each a spacer. In another example, domains s and s′ are of variable length. In another example, the spacer length (which is the length of domain s and/or domain s′) is selected to allow binding of domain x* and a domain y* of a second initiator molecule to the one or more target(s) of interest. In other words, the length of domains s and s′ are to be selected to allow for domains b₁*, b₂* and c* to be adjacent to each other, facilitated by the activation via domains a and e (that is to say, to facilitate the binding of domains a and e to domains a* and e*, respectively). This means, in order to fulfil this requirement, in some embodiments, the lengths of domains s and s′ can differ. In another example, domain s is between 1 to 50 nm long. In yet another example, domain s′ is between 1 to 50 nm long. In one example, the spacer length ranges 1 nm (roughly equivalent to a 3 thymine spacer) to 10 nm (roughly equivalent to a 30 thymine spacer).

The function of spacer sequences lies in enabling the initiators (structures I and II) to reach one another spatially. Thus, for example, bigger target molecules (that is the molecule that comprises the target binding sites domain x and domain y) may utilise a longer spacer length to enable signal generation. It is of note that the sequences between the domains are called linkers, but the sequences between domains a and x*, and domains a* and y*, are called spacers, instead of linkers.

In one example, the spacer sequences disclosed herein can partially or fully comprise nucleic acid molecules. Thus, in one example, the spacer sequences comprise thymines. In another example, the spacer sequences disclosed herein do not comprise nucleic acid molecules.

In such examples, the spacer sequences can comprise polyethylene glycol (PEG) and other hydrocarbon chains. Examples of such hydrocarbon chains can be, but are not limited to, a 3-carbon spacer, hexanediol, triethylene glycol, 8 ethylene oxide and 18-atom hexa-ethyleneglycol.

Such a spacer sequence can also be used between the other domains that make up structures I and II. As previously mentioned, the sequence between each of the domains is called a linker. In line with the function of the spacer sequence, as outlined above, a linker can likewise be used to overcome the issues or limitations directly resulting from steric hindrance or distance between structures I and II once bound to the target of interest. In one example, these linker sequences are not complementary to each other or to any other sequence within the structures disclosed herein. This could lead to over-stabilization of initiators (for example, structures I and II), which in turn would lead to signal generation even in absence of the target.

Thus, in one example, neighbouring domains in structures I and II are connected to each other via a linker. In another example, neighbouring domains in structures I and II are connected directly to each other, meaning that there are no linkers present between the neighbouring domains.

It is also noted for both spacer and linker sequences that their exact sequence (or whether it is even nucleic acid in nature or not) is immaterial. The exact length is also not material within limits apparent to a person skilled in the art. That is to say that the spacer and/or linker lengths should not be excessively long relative to the target size, as otherwise the rate at which the two initiators meet each other might be lowered unfavourably (in terms of kinetics). Thus, in one example, the total spacer length (that is, domains s and s′ taken together) can be at least as long the estimated size of the target molecule. On the other hand, in some examples, having a short linker sequence of up to 1 nm long (or up to 3 thymines in length) can facilitate to relax a crowded environment and improve circuit thermodynamics. This is particularly so around the three-way junction, i.e. the point where three DNA strands meet.

The first hairpin initiator molecule according to structure I, as disclosed herein, comprises a hairpin loop and a clamp domain, as shown in FIG. 1. These hairpin loop and claim domains comprise domains e, e*, b₂, b₂* and b₁*. That is to say, an initiator 1 known in the art used for association toehold design was modified to contain a hairpin lock. In addition, a clamp domain based on a previously disclosed domain b* (now denoted herein as domain b₂*) was extended by incorporating an additional domain e, so as to maintain the hairpin metastability in the absence of trigger event. This trigger event refers to the binding of the composition disclosed herein to one or more target(s) of interest.

As referred to herein, domains e and e* each refer to an elongation domain which represents the length that the association region is extended by upon target triggering. Domains b₂ and b₂* refer to a clamp domain taken from part of the complete toehold domain b*. It is a domain which forms part of stem of the hairpin lock (I1) to maintain its metastability in absence of target triggering. Domain b₁ refers to the remaining sequences after part of the toehold domain b is allocated as domain b₂*. Upon target triggering, hairpin initiator (HP-I1; also referred to as structure I) is opened and both domains b₁* and b₂* are exposed in its complete single stranded form, or its original domain b*. The relationship between the domains disclosed herein is also illustrated in FIG. 1 as provided herewith. Also, it is of note that the asterisks used herein denote that the respective domains are domains complementary to each other (also referred to as being reverse complementary sequences to each other when viewed in linear form in 5′ to 3′ direction), and not a new domain with independent functions.

In one example, domain b₁* forms a hairpin loop. As appreciated by the person skilled in the art, the length of domain b₁* is contingent on the length of the domain b₁* being sufficiently long in order to form the requisite hairpin loop. Thus, in another example, domain b₁* is at least 3 nucleotides long. On the other hand, formation of a large hairpin loop may influence the stability of the hairpin loop/toehold domain which is domain b₁*. Therefore, in another example, domain b₁* is between 3 nucleotides and 10 nucleotides long. By way of an explanation, for example, domain b₁* can effectively be length of domain b* subtracted by the length of domain b₂*. Therefore, the maximum length of domain b₁*is the maximum length of domain b* minus 2 (which is defined herein to be the minimum length of domain b₂*). The upper limit of a toehold length according to current understanding in the field is 12 nucleotides. As such, in one example, domain b₁* is up to 10 nucleotides in length.

As outlined above, domain b₂*, along with domain b₂, form a clamp domain, the function of which is to prevent premature opening of the hairpin loop in the absence of a trigger event. Thus, in one example, domains b₂* and b₂ are of the same length and are complementary to each other. As the function of domains b₂ and b₂* is to prevent premature opening of the hairpin loop in the absence of a trigger event, the length of domains b₂ and b₂* is considered to be sufficient when the binding (complementary or otherwise) between domains b₂ and b₂* is strong enough to prevent premature opening of the hairpin loop and yet weak enough to allow opening of the hairpin loop in the presence of a trigger event. Thus, in another example, domains b₂* and b₂ are at least 2 nucleotides in length. In another example, domain b₂* is between 2 to 6 nucleotides long. In a further example, domain b₂* (and therefore, domain b₂) is 2, 3, 4, 5, or 6 nucleotides long. In yet another example, domain b₂* is 2 or 3 nucleotides in length.

As used herein, the term “partially binding” or “partially complementary to” refers to the capability of two or more binding partners to bind to, or to be complementary to, each other. This can be in a degree that fulfils the requirement that, for example, two sequences are sufficiently connected to each other to allow further downstream function of the bound sequences. A binding between two binding partners, for example in cases where the binding partners are nucleic acid sequences, can be fully complementary (meaning that there are no mismatched nucleotides present in the bound sequence) or partially complementary (meaning that a number of mismatches are present in the bound sequence). In one example, a partially complementary sequence can refer to a sequence that has up to 5 mismatches (1, 2, 3, 4 or 5) in its sequence compared to the corresponding complementary sequence. In another example, the number of mismatches can be shown as a percentage of the complete sequence. For example, the amount of mismatches present in a sequence can be provided as 1%, 2%, 3%, 4%, 5%, 6%, 7%, 8%, 9% or 10% or up to 25% of any given sequence. A person skilled in the art would appreciate and be able to determine how many mismatches any given sequence can contain (based on, for example, the sequence length) in considering whether the referenced sequence is a partially complementary sequence.

The terms “nucleic acid sequences” and “nucleotides” can be and are used interchangeably in the present disclosure, as both terms refer to a multitude of nucleic acids within a given space.

The first hairpin initiator molecule as disclosed herein further comprises an additional elongation domain e, the function of which is to extend the effective length of the association domain upon target binding.

In one example, domains e and e* are complementary to each other. In another example, domains e and e* are of the same length. In yet another example, domains e and e* are of the same length and are complementary to each other. In a further example, domains e and e* are at least 2 nucleotides in length. In another example, domain e* or e is between 2 and 8 nucleotides long. In yet another example, domain e* or e is 2 nucleotides, 3 nucleotides, 4 nucleotides, 5 nucleotides, 6 nucleotides, 7 nucleotides or 8 nucleotides in length.

Depending of the location of domain e*, the same sequence, denoted as e* can appear multiple times. This is because, upon binding of hairpin initiator molecules to the target sequence(s), domain e* of structure II displaces domain e* of structure I. This is illustrated in FIG. 1. Thus, in one example, domain e* of structure II is at least 2 nucleotides in length and binds to domain e of structure I upon binding the target of interest.

As disclosed herein, structures I and II also compromise of an association domain a, or a*, respectively. This association domain a (found on structure I) binds to its counterpart domain a* on structure II. Because of the required binding between domains a and a*, in one example, these domains are partially or fully complementary to each other. In one example, domains a and a* are complementary to each other.

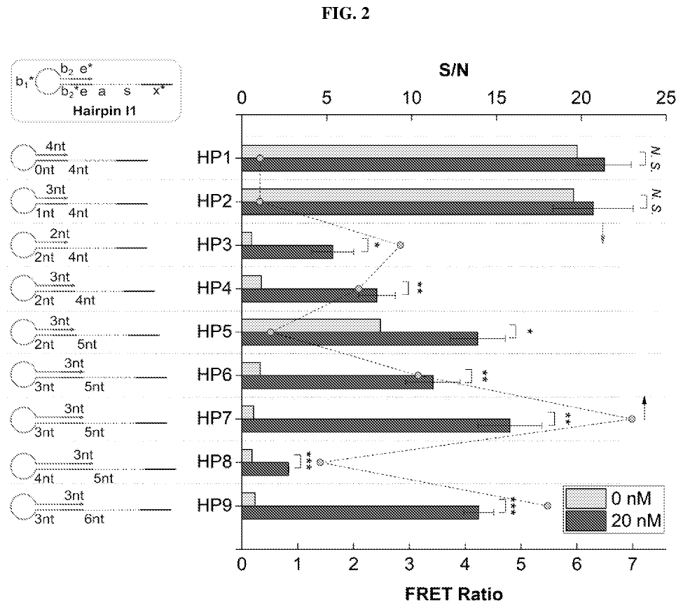

An increase in circuit leakage had been previously reported when the length of domain a reached 6 nucleotides. Thus, in order to reduce the amount of leakage which was incurred at longer association lengths, various lengths for domain a were tested, as shown for example in FIG. 2. Increasing the association length from 4 nucleotides (HP4) to 5 nucleotides (HP5) can increase the leakage further. Therefore, in one example a short association length was used resulting in low background leakage. A longer association length is understood to increase both the signal and leakage. However, when accompanied by an increased clamp length of 1 nucleotide (HP6), the Förster resonance energy transfer (FRET) signal was increased while the leakage was significantly reduced, giving a comparable signal-to-noise (S/N) ratio.

Thus, in one example, domain a is at least 3 nucleotides in length. In another example, domain a is between 3 to 10 nucleotides in length. In yet another example, domain a is 3, 4, 5, 6, 7, 8, 9, or 10 nucleotides long. In another example, domain a and domain a* are of the same length. Thus, in one example, domain a* is at least 3 nucleotides in length. In another example, domain a* is between 3 to 10 nucleotides in length. In yet another example, domain a* is 3, 4, 5, 6, 7, 8, 9, or 10 nucleotides long.

In order to be able to quantify or qualify binding of the composition disclosed herein to the one or more target(s) of interest, the composition disclosed herein can further comprise one or more structures for generating a signal, for example, by allowing binding of a signal molecule or a signal generating complex. Thus, the composition disclosed herein further comprises one or more signal generating molecules/signal generating complexes. To this end, the initiator molecule according to structure II comprises a domain c*, which, in turn, is structured or is capable of binding to a signal generating molecule/signal generating complex. This allows generation of a signal which can later be measured and from which information can be inferred. Depending on the type of signal generating molecule/signal generating complex used, domain c would be of a suitable type. That is to say, for example, if a conjugated antibody is used for signal generation, domain c* can be, but is not limited to, an antigen or an antibody binding fragment. The length of domain c* is therefore also dependent on the concept or type of signal used.

With regard to nucleic acid targets, it is reasonable to a person skilled in the art that the claimed method disclosed herein will work for detecting other nucleic acids targets (DNA and RNA) other than the ones exemplified herein, as the target recognition is facilitated by Watson-Crick base pairing. This concept is well understood, and the requisite complementary sequences to the targets of interests can be designed as required by the experimenter.

Protein detection, as referred to herein, is enabled by the binding of recognition moieties (that is domains x* and y*) to the protein of interest. Possible recognition moieties include, but are not limited to, aptamers (as exemplified by thrombin detection) and antibodies (as exemplified by Dengue antigen and IL-6), which are conjugated to the DNA probes as described in the present disclosure. Further examples as shown in Table 2 of the present disclosure. Conversely, if the target of interest is an antibody (also a type of protein), recognition moiety with binding affinity for this target class, e.g. secondary antibody or antigen, can be conjugated to the DNA probes to facilitate the detection mechanism described herein. Once the recognition moiety is able to bind to the target, the detection mechanism as disclosed herein will be able to take place. That is, the binding event leads to the initiator probes (HP-I1 and I2) being brought into physical proximity which then triggers the generation of readout signal. It is reasonable to believe that any antibody will have binding affinity for its (specific) target(s) of interest, and hence that the detection mechanism described herein can proceed.

The components and the method disclosed herein are able to detection protein with binding affinity to small molecules, as shown herein for example in Table 3. Also, the components and the method are able to detect small molecules in a competitive binding format. As an example, streptavidin is the protein which interacts with biotin, a small molecule. The small molecule is conjugated to the DNA probes and allowed to bind to the streptavidin protein. The endogenous small molecule can be added to the reaction to compete for binding to the protein and displace the currently bound small molecule modified with the DNA probe.

As an extension of one of the examples shown herein for single protein targets, it is reasonable to a person skilled in the art to extrapolate that the components and the method disclosed herein will also work for protein complexes, that is, two interacting proteins or proteins with modified regions (such as, but not limited to, phosphorylation). In one example, two binding events are used for the method and the components disclosed herein. Each DNA probe can therefore be conjugated to the recognition moiety (be in antibody, aptamer or small molecules) for each of the protein or modified region. In the protein complex form, the pair of DNA probes are then brought into physical proximity to trigger the mechanism as described herein.

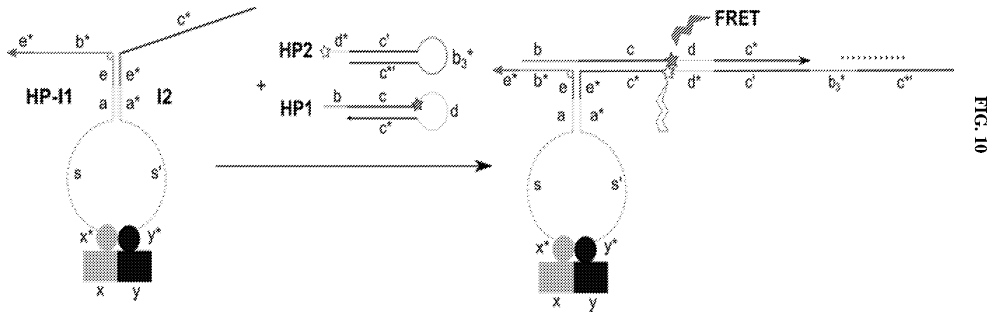

In another example, domain c* is at least the length of domain b₁*. One example where this could be the case is in the event that the readout being used was hybridization chain reaction (HCR), as shown for example in FIG. 10. In one example, where hybridization chain reaction is used, domain c* can be defined in terms of length, whereby the limitation of domain c* can be that it is at least as long as domain b1*. This requirement comes from insights gained from previous work on using hybridization chain reactions as readout methods (Ang et al., Chem. Commun. 2016, 52, 4219). The publication focusses on the HCR design, that is to say the design of HP1 and HP2 are discussed, without involvement of I1 (or currently HP-I1, disclosed herein as structure I) and I2 (disclosed as structure II herein), or the associative toehold concept, as required by the present application. In the reference publication, designing the length and CG content of the domains involved in HCR were discussed. In the present disclosure, design initiator probes to convert the target binding event into an activated form to trigger any form of compatible readout signal are disclosed, whereby one of the readout signals can be generated using HCR.

The composition disclosed herein can further comprise the use of one or more molecules for signal generation. By way of an example, the composition as disclosed herein can further comprise a pair of nucleic acid hairpins comprising a first hairpin reporter and a second hairpin reporter (structures I and II, respectively). These hairpin reporter molecules bind to the “opened” form of the composition, which is the state or form in which the structures bind to both the target of interest, as well as to each other, thereby releasing the hairpin motif made by domain b₁*, thus resulting in the generation of signal.

The target of interest of the present disclosure can be any target that a person skilled in the art would wish to detect. These targets may be found in solution, or may be on solid samples. In one example, the sample is a solid sample. In another example, the sample is a liquid sample. In another example, the target can be anchored to or embedded in a solid surface. In such cases, the method and components disclosed herein will bind to the sample, provided there is sufficient target surface protruding from the solid surface. Such examples include, but are not limited to, fixed cells, embedded cells, and cells or tissue used in imaging.

In order to do so, target recognition or binding domains x* and y* are each at least partially complementary to the one or more target of interest. In some examples, domains x* and y* are fully complementary to the one or more target of interest. In one example, domain y* is complementary, or binds to, the one or more target(s) of interest adjacent to, or downstream of, domain x. In another example, domain y* is complementary, or binds to, the one or more target(s) of interest adjacent to, or upstream of, domain x. In another example, domain x* binds, or is complementary, to the one or more target(s) of interest. In another example, the binding of the composition disclosed herein to the one or more target(s) of interest does not require enzymes.

For convention's sake, the binding sites of domains x* and y* on the target of interest are termed domains x and y, respectively.

In view of the above definition of “proximity” for structures I and II as disclosed herein, this does not necessitate that the target binding sites x and y must be next to each other in order to enable binding of structures I and II to each other. It is described herein that domains x and y are adjacent, downstream or upstream of, or in relation to, each other. In some examples, domains x and y can be adjacent to each other. This, however, does not mean to limit the physical location of domains x and y on the target of interest to be physically next to each other. For example, in the event that the target of interest is double-stranded DNA, domain x can be on the 5′ strand and domain y can be on the 3′ strand. This is irrespective of whether the target DNA is present as a double-stranded DNA molecule, or if the double-stranded DNA is presented as a denatured molecule (meaning that the 5′ strand and the 3′ strand are not bound to each other). Also encompassed in the scope of the term “adjacent” is the situation where secondary or tertiary structures of the target of interest bring domains x and y on the target of interest into proximity of each other, whereby domains x and y would not be considered to be in proximity or near to each other if the target of sequence is presented as a linear sequence (also known as the primary structure). In other words, in one example, domains x and y on the target sequence can be far apart on a protein when viewed as a linear sequence. However, folding of the protein results in domains x and y being brought close enough to each other than structures I and II as disclosed herein are able to bind to each other and to both domains x and y, respectively.

As used herein, the terms “target sequence” and “target of interest” are used synonymously and refer to a section of a target molecule which is to be detected using the methods and compositions disclosed herein. Such target(s) can be, but are not limited to, DNA, RNA, single nucleotide polymorphisms (SNP), microRNA (miRNA), genomic DNA, viral DNA, proteins, post-translational modified proteins, cell surface receptors, metabolites, lipids, carbohydrates and small molecules. In another example, the target of interest is, but is not limited to, DNA, single nucleotide polymorphisms (SNP), microRNA (miRNA), RNA, single proteins, protein complexes, small molecules, protein-small molecules, and combinations thereof. Thus, in one example, the target of interest is RNA. Examples, for RNA targets are, but are not limited to, miR-21, miR-let-7a, miR-9, miR-29, and SARS-CoV-2 or other coronaviruses. In another example, the target of interest is a single protein. Examples, for protein targets are, but are not limited to, thrombin, interleukin 6 (IL-6), and Dengue NS1. In a further example, the target of interest is a protein-small molecule, for example, streptavidin-biotin. In another example, the target of interest is a protein complex. Examples, for protein complexes are, but are not limited to, HER2/2 complex, HER2/3 complex, and HER2/2 and HER2-3 complex.

The composition disclosed herein can also be used to detect, for example, protein-protein interactions, small molecule-protein interactions, or whole cells, as shown in Table 2.

As outlined above, in order to be able to bind to said targets, the domains x* and y* are required to be molecules which are capable to binding to said targets. In other words, a person skilled in the art, having identified the target of interest, would be able to generate domains x* and y* based on the characteristics of the target of interest. Therefore, in one example, domains x* and y* are, but are not limited to, nucleic acid sequences, protein sequences, including post-translational modified versions thereof, antibodies, antigens, and small molecules.

The composition disclosed herein, that is the first hairpin initiator molecule and the second initiator molecule, and any variants thereof, can comprise nucleic acid sequences. That is to say, the domains disclosed in the respective molecules can comprise nucleic acid sequences. In one example, domains disclosed here are nucleic acid sequences. In another example, all domains, with the exception of domains x* and y*, are nucleic acid sequences. In other words, in one example, domains x* and y*, are not nucleic acid sequences. In such cases, the remainder of the structures disclosed herein can comprise or consist of nucleic acid sequences.

In one example, the composition disclosed can comprises the following domains, wherein domain a* is between 3 to 10 nucleotides in length; wherein domain b₁* is at least 3 nucleotides long; wherein domain b₂* is between 2 to 6 nucleotides long; wherein domain c* is at least the length of domains b₁*; wherein domain d* is between 6 to 12 nucleotides long; and wherein domain e* is between 2 and 8 nucleotides long. In another example, the composition disclosed herein comprises the following domains, wherein domain a* is between 3 to 10 nucleotides in length; wherein domain b₁* is at least 3 nucleotides long; wherein domain b₂* is between 2 to 6 nucleotides long; wherein domain c* is at least the length of domains b₁*; wherein domain d* is between 6 to 12 nucleotides long; and wherein domain e* is between 2 and 8 nucleotides long; and wherein domain s is between 1-50 nm long.

Also disclosed herein is a method for detecting the presence of one or more target(s) of interest in a sample, the method comprising: adding at least one first hairpin initiator molecule as defined herein and at least one second initiator molecule as defined herein to the sample thought to contain the one or more target(s) of interest (target recognition step); allowing the first hairpin initiator molecule and the second initiator molecule to bind to the target of interest, which brings the second initiator molecule into proximity with the first hairpin initiator molecule, whereby the first hairpin initiator molecule is brought into an activated form to present a three-way signal generation junction, thereby allowing binding of domains b₁* and b₂* (signal transduction step); adding at least one signal generating molecule/signal generating complex as defined herein (for example, a first reporter molecule and second reporter molecule, which can have structures according to structures III and IV, respectively), whereby binding of the at least signal generating molecule/signal generating complex generates a signal upon binding to the three-way signal generation junction; and wherein the presence of the signal indicates the presence of the one or more target(s) of interest in the sample. As used herein, the term “activated” in an activated form refers to the release of domains b₁* and b₂*, so that these domains are available for downstream hybridization reaction(s). See, for example, FIG. 1, whereby the inactivated form is represented in the second panel by the hairpin structure containing b₁* and b₂*, and the activated form is shown in the third panel by domains b* and c*.

The example outlined above illustrates one scenario using a signal generating complex using a pair of reporter molecules (also termed “reporters”). The function of these reporter molecules is to detect the binding of the composition as disclosed herein to the target(s) of interest, thereby generating a signal (also called a readout signal) which can be quantified or qualified. To this end, the signal generated can be any type of measurable signal, the most common example being an optical signal. Such optical signals can be generated directly or indirectly by using, for example, but not limited to, fluorescent molecules, fluorophores, chromophores, enzymes, proteins, dyes, pigments, conjugated antibodies, small molecules, and inorganic nanomaterials, nanoparticles and the like. As used herein, the terms “directly” and “indirectly” describe the type of binding of the reporter molecules. A direct binding indicates that a reporter molecule binds directly to the composition disclosed herein. An indirect binding indicates a reporter molecule binds to the claimed composition by way of an intermediary. Visualisation principles that can be used in the detection of a generated signal are, but are not limited to, Förster resonance energy transfer (FRET), luminescence, fluorescence, optical measurements, spectral analysis and combinations thereof. Based on the visualisation principle selected, a person skilled in the art would be able to determine the appropriate dyes, conjugates or reactants to be use in order to visualise the generated signal.

By way of an example for using gold nanoparticles (also referred to as AuNP), in their non-triggered form, HP1 and HP2 have a free single stranded end (domains b and d* respectively). The free ends bind to AuNP and stabilize the particles. The reaction solution thus remains red. When HP1 and HP2 grow into a long DNA chain from hybridisation chain reaction (HCR), the availability of free ends is reduced drastically. The gold nanoparticles aggregate when challenged with higher salt concentration to then result in a colour change in the reaction to a purple/grey colour.

As used herein, the terms “signal generating molecules” and “signal generating complexes” refer to substances which are capable in resulting in a signal output, for example, for the purpose of evaluating the results of an assay or an experiment. This readout may be the reaction of one or more signal generating molecules reacting with a given target, and/or the result of a signal generating complex reacting with a given target. A person skilled in the art would be able to select and utilise the appropriate signal generating molecule(s) and/or signal generating complex depending on the type of target or signal to be amplified. Non-limiting examples of signal generating molecules or signal generating complexes are nucleic acid sequences, proteins, antibodies, small molecules, combinations thereof and the like, and combinations or systems, such as antibody/horse-radish peroxidase detection systems and the like. In one example, hybridization chain reaction (HCR) was used as a signal generating complex, which is to say that HCR was used to generate a readout signal. In another example, Förster resonance energy transfer (FRET) is used to generate and quantify the readout signal.

As used herein, the term “detection moiety” refers to a moiety present on the signal generating molecule(s), and which is capable of generating a signal or readout upon binding of the signal generating molecule to the target. Non-limiting examples of a detection moiety are fluorophores, chromophores, small molecules, proteins, inorganic nanomaterials, and combinations thereof. In another example, a detection moiety can comprise a pair of molecules, for example a fluorophore-quencher pair. When present, the detection moiety can be found anywhere along any one of the reporter molecules as disclosed herein, so long as it fulfils its purpose of enabling detection of the triggered construct. Also, a reporter molecule can comprise one or more detection moieties. In one example, a detection moiety can be found between domains b, c and d. In another example, a detection moiety can be found at the free end of domain d* or along any other domains, for example, domain c′. In one example, a reporter molecule comprises 2 detection moieties. Also included herein are examples in which reporter molecules do not comprise any detection moieties. In such examples, binding of the signal generating molecule is done using other means, for example, but not limited to, changes in the structure or any other characteristic of the target molecule itself.

In one example, the composition disclosed herein further comprises one or more reporter molecules. In one example, the reporter molecules as disclosed herein are used for signal generation and/or amplification. In another example, the composition disclosed herein comprises a pair of reporter molecules. In yet another example, the reporter molecules are a first hairpin reporter and a second hairpin reporter. Examples of structures of hairpin reporters can be found in HP1 and HP2 as disclosed herein. In another example, the reporter molecules are nucleic acid hairpins comprising according to structures III and IV, respectively:

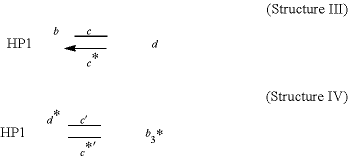

In this example, the first hairpin reporter comprises domains b, c, d and c*. In one example, domain b is a 5′ nucleic acid overhang with a length between 6 to 12 nucleotides. In another example, domain d is a hairpin loop with a length between 6 to 12 nucleotides. In one example, the first hairpin reporter comprises a first detection moiety; optionally between domains c′ and d. In another example, domain c* of structure III is as defined herein. In yet another example, domain b is a 5′ nucleic acid overhang with a length between 6 to 12 nucleotides, domain d is a hairpin loop with a length between 6 to 12 nucleotides, and domain c* is as herein.

In another example, the second hairpin reporter comprises domains d*, c′, b₃* and c*′. In one example, domain b₃* is a hairpin loop of the same length as domain b. In one example, domain b₃* is at least partially complementary to domain b. This is because once bound to the hairpin initiator molecule in triggered state, the first and second hairpin reporters unfold and bind to the hairpin initiator molecule, as shown for example in FIG. 10.

In another example, domain d* is a nucleic acid overhang of the same length as domain d. In a further example, domain d* is between 6 to 12 nucleotides long. In yet another example, domains c′ and c*′ are of the same length, and are capable of binding to each other. In a further example, domains b₃* and d each form a hairpin loop. In another example, domain d and d* are complementary to each other. In one example, the domains c′ and c*′ are complementary to each other. In another example, the second hairpin reporter comprises a second detection moiety; optionally at the free end of domain d* or along domain c′. In yet another example, domain b₃* is a hairpin loop of the same length as domain b, wherein domain b₃* is at least partially complementary to domain b, domain d* is a nucleic acid overhang of the same length as domain d, domains c′ and c*′ are of the same length, and are capable of binding to each other, and domains b₃* and d each form a hairpin loop.

Thus, in one example, the first hairpin reporter comprises domains b, c, d and c*; domain b is a 5′ nucleic acid overhang with a length between 6 to 12 nucleotides; domain d is a hairpin loop with a length between 6 to 12 nucleotides; the second hairpin reporter comprises domains d*, c′, b₃* and c*′; domain b₃* is a hairpin loop of the same length as domain b, wherein domain b₃* is at least partially complementary to domain b; domain d* is a nucleic acid overhang of the same length as domain d; domains c′ and c*′ are of the same length, and are capable of binding to each other; and domains b₃* and d each form a hairpin loop.

With regard to structure of the reporter molecules, for example, domains b* and d* are toeholds, which are accepted in the art to having a length in the range of between 6 to 12 nucleotides. The inventors had previously found through simulation and experimental validation (data not shown) that the stem length, or domain c*, be at least as long as the toehold length, in order to stabilize the hairpin structure.

In one example, the composition disclosed herein comprises one or more of the nucleic acid sequences as defined in, but not limited to, SEQ ID NO: 1 to 77. In another example, comprises one or more of the nucleic acid sequences as defined in, but not limited to, SEQ ID NO: 28 to 45. In another example, the composition comprises any one or more of the following pairs: SEQ ID NOs. 28 and 29, SEQ ID NOs. 30 and 31, SEQ ID NOs. 32 and 33, SEQ ID NOs. 34 and 35, SEQ ID NOs. 36 and 37, SEQ ID NOs. 38 and 39, SEQ ID NOs. 40 and 41, SEQ ID NOs. 42 and 43, and SEQ ID NOs. 44 and 45. In a further example, the composition comprises any one or more of the following pairs: SEQ ID NOs. 28 and 29, SEQ ID NOs. 42 and 43, and SEQ ID NOs. 44 and 45.

Further, non-limiting examples of signal generating molecules/signal generating complexes can be found in the section below.

For hybridization chain reaction (HCR) using Förster resonance energy transfer (FRET) readout:

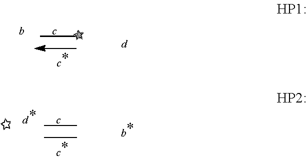

It is noted that the position of the fluorophores (indicated by stars in the schematic above) are not fixed, and can vary anywhere along the entire sequence, so long as the relative position of the stars to each other generates a readable signal over noise.

For HCR using HRP-mimicking DNAzyme:

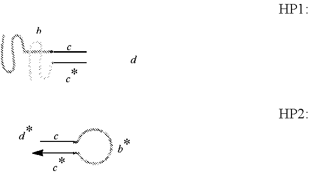

An exemplary DNA sequence which folds into secondary structure with HRP-mimicking catalytic function is split into two entities and appended at the ends of domains b and c* of HP1 or d* and c* of HP2. Upon activation, the split sequences reunite to form the complete DNAzyme which catalyses the formation of readout signals.

For HCR using split enzyme motif:

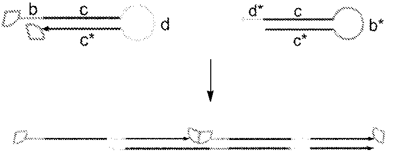

Catalytic enzymes, such as for example beta-galactosidase, can be split into two entities, which recombine only upon signal activation. In such a situation, split beta-galactosidase can be conjugated onto hairpin monomers to generate this effect.

For fluorophore-quencher (F-Q) readout:

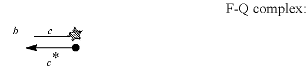

It is noted that the fluorophore described here is represented by the star and quencher is represented by a circle, and they can be swapped between the two strands. Their positions can vary anywhere along the domain c, as long as they are adjacent to each other.

For X-probe readout:

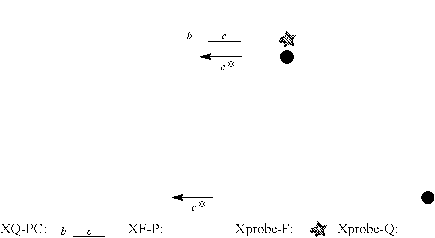

X-probe can be used, wherein Xprobe-F and Xprobe-Q are universal sequences, and the sequences of XQ-PC and XF-P strands can be varied accordingly. This allows the labelled oligonucleotides to be reused regardless of the target/probe sequences used, representing cost savings and reduction in turnaround time during probe design optimization.

Non-limiting examples of duplex and/or hairpin structures

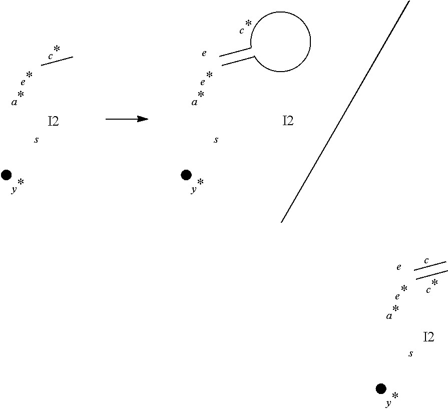

The ratio of component strands used in the exemplary method disclosed herein can be varied. For example, ratios of HP1:HP2 between 1:1 to 2:1 can be used and is dependent on the intended target. For example, when detecting nucleic acid targets, the ratio of HP-I1 and I2 can be 1:1. In other examples, the relative ratios of HP-I1 and I2 for other target classes depends on the relative binding affinity of the recognition moieties x* and y*. The relative binding affinity of the recognition moiety x* and y* will differ according to the specific target (for example, one can use the relative dilution ratio recommended for commercial antibodies when detecting protein targets). The ratio of [HP1/HP2] to [HP-I1/I2] taken as a group, that is to say that the ratio of readout strands to initiator strands, ranges from 1:1 (typically used) to 5:1 (can be used to improve assay kinetics). The absolute concentration can be used from sub-nanomolar to micromolar ranges. In one example, a ratio is 1:1:1:1 for all 4 components is used, with the explicit concentration being 20 nM. For the experimental results shown in this example, a ratio of 1:1:2:1 (Structure I-IV, in order) had been used, with the explicit concentration being 20 nM HP-I1, 20 nM 12, 40 nM HP1 and 20 nM HP2. However, as a person skilled in the art will appreciate, these concentrations and ratios can be optimised to requirements dictate by the intended target of the method disclosed herein.

In one example, the composition as disclosed herein comprises a domain a of 4 nucleotides, a domain b₁ of 9 nucleotides, a domain b₂ of 2 nucleotides, a domain c of 12 nucleotides, a domain d of 6 nucleotides, a domain e of 2 nucleotides, and a domain s of 15 nucleotides in length (about roughly 5 nm in length). In another example, the composition as disclosed herein comprises a domain a of 4 nucleotides, a domain b₁ of 9 nucleotides, a domain b₂ of 3 nucleotides, a domain c of 12 nucleotides, a domain d of 6 nucleotides, a domain e of 2 nucleotides, and a domain s of 15 nucleotides (about roughly 5 nm in length). In yet another example, the composition as disclosed herein comprises a domain a of 5 nucleotides, a domain b₁ of 9 nucleotides, a domain b₂ of 3 nucleotides, a domain c of 12 nucleotides, a domain d of 6 nucleotides, a domain e of 2 nucleotides, and a domain s of 15 nucleotides in length (about roughly 5 nm in length). In yet another example, the composition as disclosed herein comprises a domain a of 5 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 15 nucleotides in length (about roughly 5 nm in length). In yet another example, the composition as disclosed herein comprises a domain a of 5 nucleotides, a domain b₁ of 9 nucleotides, a domain b₂ of 4 nucleotides, a domain c of 12 nucleotides, a domain d of 6 nucleotides, a domain e of 3 nucleotides, and a domain s of 15 nucleotides in length (about roughly 5 nm in length). In yet another example, the composition as disclosed herein comprises a domain a of 6 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 15 nucleotides in length (about roughly 5 nm in length). In yet another example, the composition as disclosed herein comprises a domain a of 6 nucleotides, a domain b₁ of 9 nucleotides, a domain b₂ of 4 nucleotides, a domain c of 12 nucleotides, a domain d of 6 nucleotides, a domain e of 5 nucleotides, and a domain s of 15 nucleotides in length (about roughly 5 nm in length). In yet another example, the composition as disclosed herein comprises a domain a of 8 nucleotides, a domain b₁ of 9 nucleotides, a domain b₂ of 5 nucleotides, a domain c of 12 nucleotides, a domain d of 6 nucleotides, a domain e of 5 nucleotides, and a domain s of 15 nucleotides in length (about roughly 5 nm in length). In yet another example, the composition as disclosed herein comprises a domain a of 6 nucleotides, a domain b₁ of 9 nucleotides, a domain b₂ of 5 nucleotides, a domain c of 12 nucleotides, a domain d of 6 nucleotides, a domain e of 5 nucleotides, and a domain s of 15 nucleotides in length (about roughly 5 nm in length). In yet another example, the composition as disclosed herein comprises a domain a of 6 nucleotides, a domain b₁ of 9 nucleotides, a domain b₂ of 6 nucleotides, a domain c of 12 nucleotides, a domain d of 6 nucleotides, a domain e of 5 nucleotides, and a domain s of 15 nucleotides in length (about roughly 5 nm in length). In yet another example, the composition as disclosed herein comprises a domain a of 6 nucleotides, a domain b₁ of 9 nucleotides, a domain b₂ of 5 nucleotides, a domain c of 12 nucleotides, a domain d of 6 nucleotides, a domain e of 6 nucleotides, and a domain s of 15 nucleotides in length (about roughly 5 nm in length). In yet another example, the composition as disclosed herein comprises a domain a of 4 nucleotides, a domain b₁ of 9 nucleotides, a domain b₂ of 5 nucleotides, a domain c of 12 nucleotides, a domain d of 6 nucleotides, a domain e of 8 nucleotides, and a domain s of 15 nucleotides in length (about roughly 5 nm in length). In yet another example, the composition as disclosed herein comprises a domain a of 6 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 15 nucleotides in length (about roughly 5 nm in length). In yet another example, the composition as disclosed herein comprises a domain a of 5 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 6 nucleotides in length (about roughly 2 nm in length). In yet another example, the composition as disclosed herein comprises a domain a of 5 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 6 nucleotides in length (about roughly 2 nm in length). In yet another example, the composition as disclosed herein comprises a domain a of 5 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 6 nucleotides in length (about roughly 2 nm in length). In yet another example, the composition as disclosed herein comprises a domain a of 5 nucleotides, a domain b₁ of 9 nucleotides, a domain b₂ of 3 nucleotides, a domain c of 12 nucleotides, a domain d of 6 nucleotides, a domain e of 3 nucleotides, and a domain s of 6 nucleotides in length (about roughly 2 nm in length). In yet another example, the composition as disclosed herein comprises a domain a of 4 nucleotides, a domain b₁ of 9 nucleotides, a domain b₂ of 2 nucleotides, a domain c of 12 nucleotides, a domain d of 6 nucleotides, a domain e of 2 nucleotides, and a domain s of 15 nucleotides in length (about roughly 5 nm in length). In yet another example, the composition as disclosed herein comprises a domain a of 4 nucleotides, a domain b₁ of 9 nucleotides, a domain b₂ of 2 nucleotides, a domain c of 11 nucleotides, a domain d of 6 nucleotides, a domain e of 2 nucleotides, and a domain s of 15 nucleotides in length (about roughly 5 nm in length). In yet another example, the composition as disclosed herein comprises a domain b₁ of 9 nucleotides, and a domain c of 12 nucleotides. In yet another example, the composition as disclosed herein comprises a domain b of 9 nucleotides, a domain c of 12 nucleotides, and a domain d of 6 nucleotides. In a further example, the composition as disclosed herein comprises a domain a of 3 nucleotides, a domain b₁ of 7 nucleotides, a domain b₂ of 2 nucleotides, a domain c of 12 nucleotides, a domain d of 9 nucleotides, a domain e of 3 nucleotides, and a domain s of 15 nucleotides (about roughly 5 nm in length).

In view of the disclosure herein, in the event that further sequences required by the structures disclose herein are not explicitly mentioned (for example, sequence k is defined but sequence k* is not defined), a person skilled in the art will appreciate that the asterisks domains are the reverse complementary versions (when viewed in 5′ to 3′ direction) of the corresponding non-asterisk domain, which are complementary in binding to the corresponding non-asterisk domain, and need not be explicitly mentioned. In one example, the asterisk domain same is length as the non-asterisks counterpart. An exception to this guide is domain x*, as the length of domain x and domain x* can be different. This is because the length of domain x* depends on the binding site, domain x, on the target of interest. That is to say, the length of domain x* can be shorter than the length of domain x. This can also apply to domains y and y*, whereby the length of domain y* can be shorter than the length of domain y, as illustrated for domain x above.

In one example, wherein each component of one or more compositions, as defined herein, is added simultaneously or sequentially. In another example, all components of the compositions disclosed herein can be added all at the same time, along with a sample containing the one or more target(s) of interest, thereby resulting in a so-called “one-pot reaction”. Thus, in another example, the method as disclosed herein is performed in a single reaction vessel.

The elongated association toehold concept utilized a hairpin lock to dynamically tune the association region length from an initial shorter length to a final longer length when triggered. As such, this design was able to show improve kinetics and thermodynamics from having a longer association region, without the trade-off of incurring higher circuit leakage rate. A detailed study was carried out to understand the contributions each additional domains in this modified design, which culminated in a set a design guidelines. Three hairpin-locked initiator designs with elongated association lengths of 4→6 nucleotides, 5→8 nucleotides and 6→9 nucleotides were used to characterize the impact of this design on the equilibrium signal (thermodynamics) and overall circuit kinetics. It was demonstrated that the dynamically elongated association toehold design boosted the equilibrium signal many fold, with its kinetics approaching close to the upper limit possible for each final association length, while the hairpin lock was effective in suppressing the circuit leakage. This translated to improved practical performance with lowered limit of detection and increased detection speed.

Also disclosed herein is a method for detecting one or more target(s) of interest in a sample, the method comprising providing a sample thought to comprise the one or more target(s) of interest and detecting the one or more target(s) of interest using the composition as disclosed herein, whereby each composition is specific for one target of interest. In another example, the composition disclosed herein can be specific for more than one target of interest. In yet another example, the composition can bind to more than one target of interest simultaneously. In a further example, the first hairpin initiator molecule and the second initiator molecule, as defined herein, bind to at least one target(s) of interest.

In another example, there is disclosed a method for detecting the presence of one or more target(s) of interest in a sample, the method comprising adding one or more compositions as disclosed herein to the sample; allowing binding of the one or more compositions to the one or more target(s) of interest thought to be comprised in the sample; measuring one or more signals resulting from the binding of the composition(s) to the target(s) of interest; wherein the generation of one or more signals detects the presence of one or more of the target(s) of interest in the sample.

It is also illustrated herein that the methods disclosed herein are performed at a single temperature (that is, an isothermal method), as opposed to methods requiring multiple temperatures known in the art. In one example, the method as disclosed herein was performed at a single temperature. In another example, the temperature at which the method disclosed herein is performed is a temperature at which the resulting complex (that is a complex comprising the target of interest, the first hairpin initiator molecule and the second initiator molecule) is stable. In another example, the method disclosed herein is performed at room temperature. In another example, the method is performed at a single temperature, at which the composition(s) bound to the target(s) of interest are stable. That is to say that the method disclosed herein can be performed at a temperature of up to 60° C. Experimental settings beyond 60° C. would require longer domain lengths in order to ensure the complex stability.

The underlying explanation is that, in some examples, the method is driven by DNA hybridization. Based on this it can be said that DNA complexes will remain stable up to 60° C., a common annealing temperature used in polymerase chain reactions (PCR), a method which is also known to be based on DNA hybridization. The stability of DNA hybridization is indicated by its melting temperature, which in turn is determined by its length of, for example, the primer used. The primer lengths are typically, but not limited to, 18 to 22 nucleotides, while the combined length of domains b* and c*, responsible for driving the signal generation step, is greater than 18 nucleotides for the shortest design example disclosed herein. The other domains a and e (elongated association length), or e and b₂* (stem of hairpin lock in HP-I1 (also referred to as structure I)), are comparatively shorter. However, they exist in high local concentration, i.e. the target bringing HP-I1 and I2 (structures I and II, respectively) together in close proximity for the former (that is, domains a and e) and within a hairpin structure for the latter (that is, domains e and b₂*). Their structures, be it in duplex at high concentration for domains a and e or in a hairpin structure for domains e and b₂*, remain intact at temperatures up to 60° C., even for the shortest example length of 4 nucleotides used in the simulation. Here, the shortest length of 4 nucleotides refers to when domain e and b₂* are each 2 nucleotides long, for a total length of 4 nucleotides.

The methods disclosed herein are performed on samples thought to contain the one or more target(s) of interest. These samples can be, but are not limited to biological samples and non-biological samples. Non-limiting examples of biological samples are, but are not limited to, any quantity of a substance from a living thing or formerly living thing. Such living things include, but are not limited to, humans, mice, monkeys, rats, rabbits, and other animals. Such substances include, but are not limited to, blood, serum, plasma, urine, cells, organs, tissue, diseased tissue, bone, bone marrow, lymph, lymph nodes, endothelial cells, vascular tissue, and skin. Non-limiting samples of non-biological samples include, but are not limited to, drug samples, fluid samples and samples that have not been obtained from a living thing or formerly living thing. In another example, a sample can also contain bacteria, virus, fungi and the like.

Also disclosed herein is the use of the composition or the method disclosed herein in drug screening and/or drug discovery. This would be an example of use of the composition described herein on a non-biological sample. In such an example, the method as disclosed herein can be used to measure interaction between the drug and target of interest. By way of an example using cell surface markers as target, one of the initiator probes can be conjugated onto the antibody or small molecule drug candidate, while the other initiator probe can be conjugated to an antibody specific to the target of interest, e.g. cell surface receptor protein. When the drug candidate binds to the target of interest, a signal is generated by mechanism of the method described herein. By way of another example using a solution phase, some drug discovery platforms or methods involve measurements of cytokines (which are protein targets), which are secreted by cells when these come into contact with drugs. The method disclosed herein can be used in drug discovery to quantify the level of cytokine secretion of the cells in real-time, as an effect of coming into contact with one or more drugs.

Also disclosed herein is a kit comprising the composition as defined herein. This kit is to be utilised according to the method disclosed herein. Thus, in one example, the kit comprises at least structures I, II, a first reporter molecule and a second reporter molecule as disclosed herein. In another example, these components are kept separately. In another example, the kit as disclosed herein comprises a first hairpin initiator molecule and a second initiator molecule, wherein the initiator molecules are further modified to be suitable for conjugation. In another example, the kit optionally comprises i) reagents for performing affinity conjugation, or ii) reagents for performing covalent conjugation with a molecule selected from, but not limited to, DNA, antibody and proteins.

In one example, in the kit as described herein, each component of the composition is provided separately, in the form of a lyophilised powder or as a concentrated stock solution. Which form the component is provided in depends on various considerations, the main consideration being the stability of the components. For example, for easier handling and less reliance on a cold chain, DNA and RNA can be provided as lyophilized powders, which are then reconstituted with water or a suitable buffer prior to use.

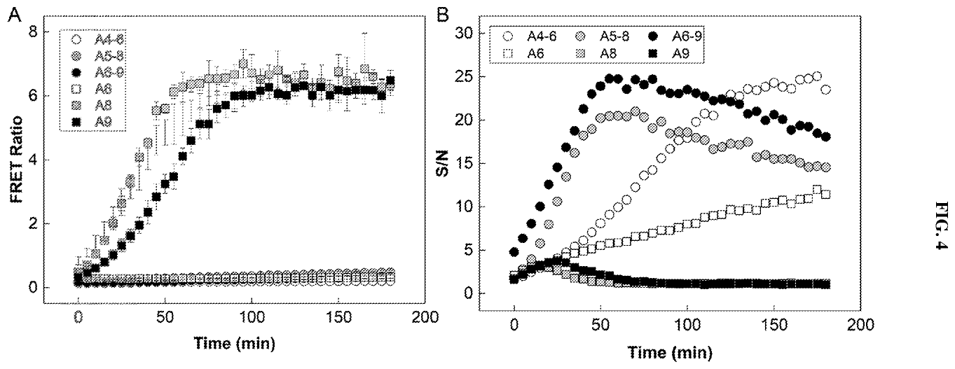

To arrive at the composition disclosed herein, design guidelines were established for the stable elongation of association toehold, and it was ascertained how different configuration of the modified domains impacted the overall performance of the proposed design. Three sets of hairpin initiators involving different extent of association length elongation, effectively, the sum of domains a and e, or domains a* and e*, respectively. (4→6 nucleotides, 5→8 nucleotides and 6→9 nucleotides) were shortlisted from the design framework. Direct experimental evidence of these hairpin initiator designs in improving the circuit performance without the penalty of incurring substantial circuit leakage has been determined using examples of 4→6 nucleotides, 5→8 nucleotides and 6→9 nucleotides (length of domain a 4 combined length of domain and e) (see FIGS. 4 to 6). The circuit performance was evaluated based on reaction speed, equilibrium state attained and limit of detection.

### Considerations for Designing Dynamically Elongated Association Toehold

A difficulty to designing a dynamically elongated association toehold which needed to be overcome was to have good balance of the domain configurations on the hairpin-modified initiator 1 (HP-I1). HP-I1 differed from the conventional I1 design by i) an additional elongation domain e and ii) a domain b₂* clamp originating from part of the toehold domain b. As with any circuit design, the foremost consideration was to ensure that the circuit did not leak substantially. In this case, the leak condition depended on whether the hairpin lock (“closed” state defined by domain b₂* and e) was able to protect the elongation domain e from exposing itself (“opened” state defined by domains a and e) in absence of triggering event.

Different combinations of domains a, e and b₂* in HP-I1 were compared in FIG. 2, with domains a and e having an equal length of 4 nucleotides each. Starting from an association length of 4 nucleotides, in one example, an equal length of domain e was added which was also the minimal length for a stable hairpin loop design. This leakage was observed (HP1) and did not subside (HP2) until 2 nucleotides of domain e was removed and replaced with a 2 nucleotide clamp in the form of domain b₂* (HP3).

It is noted that the above paragraph refers to the hairpin stem (not loop), which comprises 4 nucleotides. The length of the hairpin stem on HP-I1 (for example, structure I) is the combined length of domains b₂* and e, which is independent of the length of domain b₁* (hairpin loop). This is also consistent with the defined range of domain b₂* and e as disclosed herein, where each of the two domain has to be minimally 2 nucleotides long, making it a total of 4 nucleotides which are used in this example to stabilize the stem of the hairpin.

In this context, the clamp served as a buffer domain to avoid the contradictory scenario of having domain e as both the branch migration domain of the association step (favours “opened” state) and the entire hairpin stem (favours “closed” state), which proved to be detrimental to circuit leakage (as seen in design HP1). Part of the toehold domain b was used as the clamp domain to avoid introducing additional gaps between the c* b* trigger domains upon association (FIG. 1). With the clamp domain in place, it was attempted to boost the equilibrium signal by increasing the elongation domain e by 1 nucleotide in design HP4, which instead led to a decrease in “signal to noise” (S/N) ratio due to increased leakage over time.

An increase in circuit leakage had been previously reported when the length of association domain a reached 6 nucleotides, at least within the reaction condition of the study and using the specific set of sequences. It was investigated if it were possible to reduce the amount of leakage incurred at longer association lengths with the presence of a hairpin lock as a form of competing hybridization reaction. Increasing the association length from 4 nucleotides (HP4) to 5 nucleotides (HP5) expectedly worsened the leakage further. However, when accompanied by an increased clamp length of 1 nucleotide (HP6), the Förster resonance energy transfer (FRET) signal was boosted while the leakage was significantly reduced, giving a comparable signal-to-noise (S/N) ratio as the least leaky design for a 4-nucleotide association length (HP3).

It was then questioned if the signal-to-noise (S/N) ratio could be further improved, either through sequence redesign or by increasing the clamp length (to further suppress the leakage). The elongation domain e was changed from GGC (HP6) to GTG (HP7), which was thought to weaken the reactions favouring both the “closed” and “opened” state. Interestingly, this modification resulted in a net increase in Förster resonance energy transfer (FRET) signal and reduction of circuit leakage, indicating that the sequence design of domain e played a more prominent role as the branch migration domain than as part of the hairpin stem. When the clamp length (that is, domains b₂ and b₂*, respectively) was increased by 1 nucleotide to 4 nucleotides (HP8), both the signal and leakage dropped significantly. This suggested that excessive hairpin stem length will impede the association reaction, especially if it were due to long clamp length which played no role in promoting the “opened” state. Thus, in one example, the clamp length (defined by domains b₂* and b₂) was kept at 2 to 3 nucleotides for the examples shown herein. In another example, the length of domains b₂ and b₂* is between 2 to 6 nucleotides, or 2, 3, 4, 5, or 6 nucleotides.

With this understanding of factors affecting the design of HP-I1, it was possible to design an elongated association toehold for an initial association length of 6 nucleotides (HP9), which would have otherwise been the most challenging design requirement. The observations made can be summarised into following design guidelines:

In one example, the clamp domain b₂* can be used for a stable “closed” state. In another example, the clamp domain b₂* is 2 to 6 nucleotides long.

In another example, the elongation domain e is between 2 and 8 nucleotides in length. In another example, the elongation domain e is 2, 3, 4, 5, 6, 7 or 8 nucleotides in length.

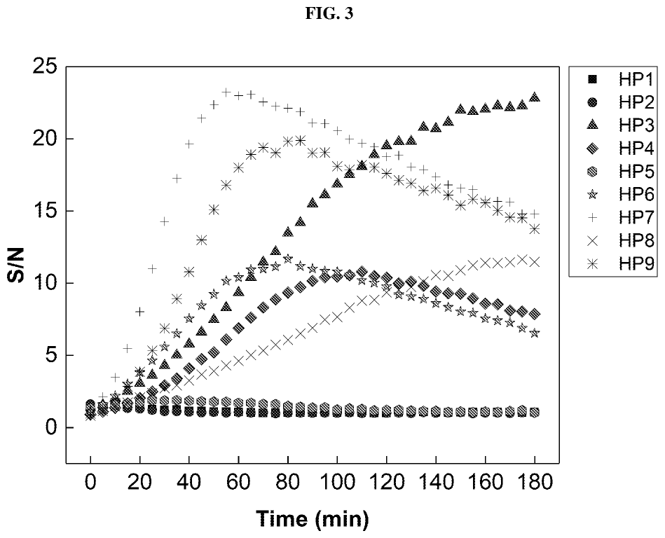

One of the designs disclosed herein, for example HP3 (denoted as A4-6 from this point which represented the elongation of an initial association length of 4 nucleotides to the final effective length of 6 nucleotides), HP7 (A5-8) and HP9 (A6-9), were shortlisted for a more thorough comparative study between the conventional association toehold and dynamically elongated association toehold design in the next sections (FIG. 3).

### Improved Control of Circuit Leakage

The premise of the proposed elongated association toehold was to reconcile the trade-offs between the rates of desired reaction and leakage. As such, it will only be considered an applicable concept if the circuit can benefit from improved reaction rates with a longer association region without incurring significant leakages and consequently background noise. To test these criteria, the extent of leakage and its impact on signal-to-noise (S/N) between the HP-I1 and conventional I1 design with an association length equal to the final, elongated association length in HP-I1 were compared.

Significant leakage was incurred when using long association lengths of 8 nucleotides and 9 nucleotides, which was suppressed drastically using the dynamically elongated association toehold concept (FIG. 4A). This translated into obvious significant improvement in the signal-to-noise (S/N) ratio of the circuit (FIG. 4B). The signal-to-noise ratio of the elongated association toehold system decreased gradually over time after 60 minutes due to a steady but much slower leakage reaction. Moreover, it should be kept in mind that equilibrium signal was obtained rapidly (within 60 min) using the elongated association toehold. Given a constant signal and a slow evolution of noise in the form of leakage, the signal-to-noise ratio will mathematically decline after the signal equilibrium point. In another words, the structures disclosed herein makes use of a longer association region for improved signal generation yet without incurring significant leakage.

### Improved Kinetics and Thermodynamics Using Elongated Association Toehold

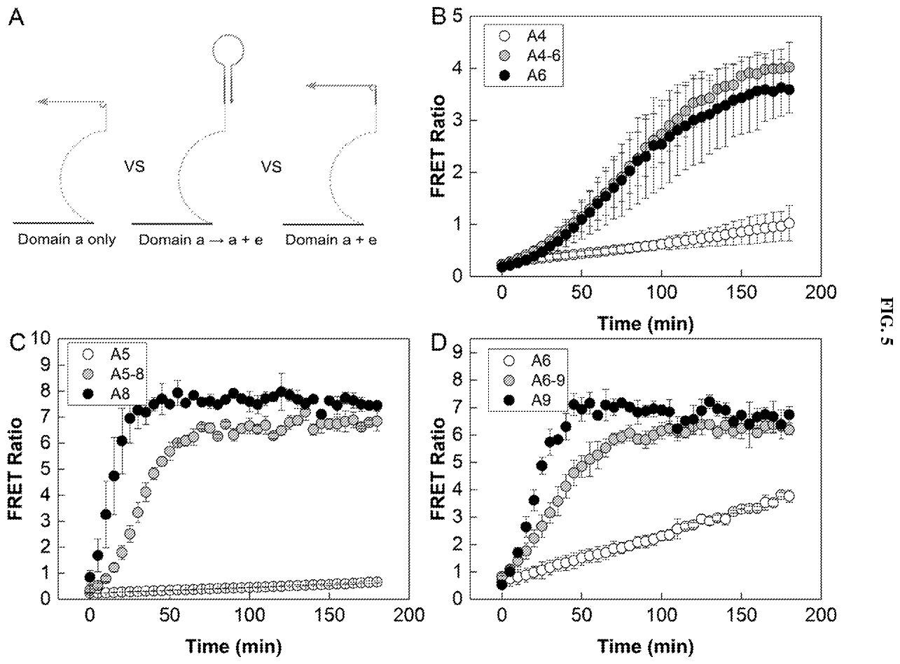

Next, it was investigated if the elongated association toehold benefited from improved kinetics and thermodynamics, as hypothesized from the final, longer association toehold length. That is, that the additional kinetic step from the opening of HP-I1 should not negatively impair the overall circuit performance. The evolution of Förster resonance energy transfer (FRET) signal in presence of split trigger (ST) in the elongated association toehold design (domain a→a and e) was compared with the conventional association toehold design with both the initial (domain a only) and final (domain a and e) association length (FIG. 5A).

The elongated association toehold design outperformed the conventional design with initial association length, both in terms of the amount of equilibrium signal obtained and reaction kinetics (FIG. 5B-D). Its equilibrium signal approached close to that achieved by the final elongated association length design, which was within expectation as the final ST-I1-I2 assembly was almost equivalent in both designs (except for the domain e* overhang in HP-I1). The circuit kinetics was slower than the longer association length design, which was understandable given the additional hairpin lock meant that additional kinetics, and often, competing step(s) were involved in this elongated association toehold design. In another words, the hairpin initiator design improved signal generation compared to the initial, shorter association region in a linear initiator design, which now came close to the performance of the longer association region in a linear initiator design but without incurring the circuit leakage.

### Improved Analytical Performance

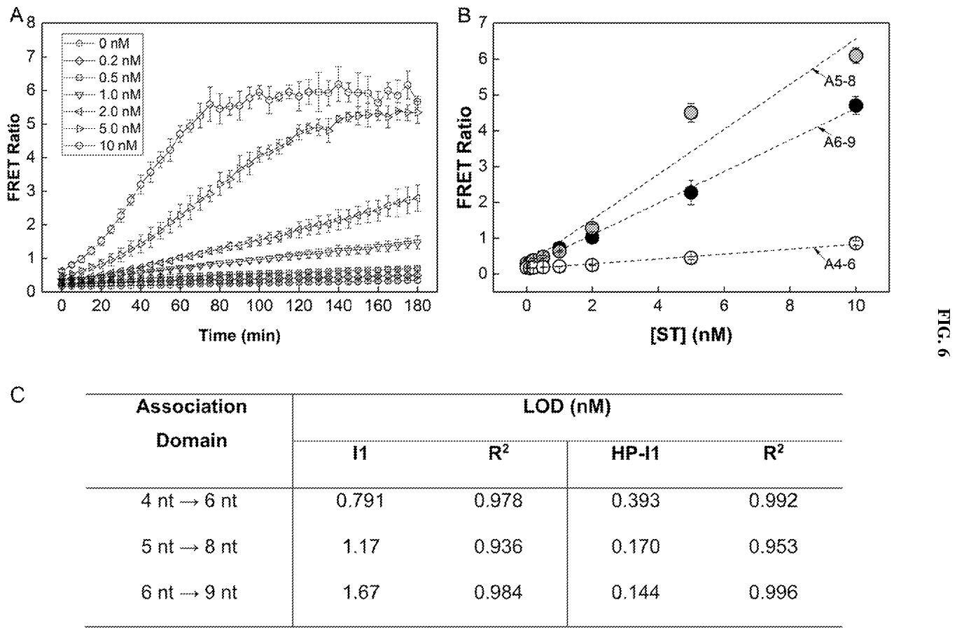

The analytical performance of split proximity circuits (SPC) were analysed using the dynamically elongated association domain to assess if its superior kinetics and thermodynamics could be translated into practical applications. The Förster resonance energy transfer (FRET) signal generated from the modified split proximity circuit was dependent on the concentration of split trigger (ST) titrated (FIG. 6A). Linear dosage relationship was observed for the concentration range ca. 0.2 to 10 nM (FIG. 6B). This dynamic range was in line with previous work on hybridization chain reaction (HCR), the readout signal of which used in the experiments herein. The hairpin initiator generated quantifiable signal with direct correlation to the target concentration.

The performance of the modified split proximity circuit design was compared to designs known in the art (see Ang et al., 2016) based on the shorter initial association length by evaluating the limit of detection (LOD) in the two designs for the three association lengths of 4-6 nucleotides after an hour of reaction (Table 2). It is noted that it was not meaningful to compare with the longer final association length due to the extensive amount of leakage. The limits of detection in the modified split proximity circuit were on average, an order of magnitude, improved over split proximity circuits known in the art (see Ang et al., 2016). It was further noticed that the limit of detection improved with increasing association length for the HP-I1 design, which was otherwise considered to be detrimental for the conventional design, due to the increasing extent of leakage with association length. This limit of detection can be further improved by optimizing the concentrations of the split proximity circuit (SPC) reactants. Here, the hairpin initiator design increased the assay sensitivity, which was measured in terms of the limit of detection.

This improved performance was attributed to a combination of different factors, for example, increased circuit kinetics leading to the generation of a substantially greater amount of Förster resonance energy transfer (FRET) signal, which outweighed the effects of the much slower leakage reaction. A caveat to attaining equilibrium signal within a much shorter time frame is that analysis be kept short (for example, to within an hour); otherwise the boost in signal-to-noise ratio arising from the improved kinetics and thermodynamics would soon be eroded by the gradual increase in circuit leakage. The ability to perform within a short time frame is considered to be an advantageous feature for applications considered within the scope of the present disclosure. In another words, the hairpin initiator generated a stronger signal within a shorter time for improved target detection.

TABLE 1
Comparison of the limit of detection (LOD) achieved at t = 60 minutes
using an initiator 1 (I1) known in the art (see Ang et al., 2016) and
hairpin initiator 1 (HP-I1) designs. The initial, shorter association
domain length was used to characterize the I1 system.

| Association | LOD (nM) | | | |
|---|---|---|---|---|
| Domain | IL | R² | HP-I1 | R² |
| 4 nt → 6 nt | 0.791 | 0.978 | 0.393 | 0.992 |
| 5 nt → 8 nt | 1.17 | 0.936 | 0.170 | 0.953 |
| 6 nt → 9 nt | 1.67 | 0.984 | 0.144 | 0.996 |

nt-nucleotides

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

All DNA and RNA oligonucleotides used in this study were purchased from Integrated DNA Technologies (IDT), and HPLC purified by IDT unless otherwise stated. The nucleic acid sequences are shown in Table 3. The lyophilized DNA was reconstituted in 1× Tris-EDTA buffer (1×TE, pH 8.0) to give ca. 100 μM stock and stored at 4° C. for up to a year, except for Cy3/Cy5-modified DNAs which were stored at −20° C. and protected from light. The lyophilized DNA was reconstituted in nuclease-free water to give ca. 100 μM aliquot stock and stored at −80° C. The following chemicals were used as received: sodium chloride (NaCl, ≥99.5%) and magnesium chloride (MgCl₂, ≥98%) were purchased from Sigma Aldrich. 10× phosphate buffered saline (PBS, pH 7.4) and 1× Tris-ETDA (TE, pH 8.0) was purchased from 1st BASE. Milli-Q water with resistance >18.2 MP/cm was used throughout the experiment.

### NUPACK Simulation

NUPACK web server was used for the design and analysis of nucleic acid structures and systems. Nupack analysis was carried out for n number of interacting DNA species at 25° C. to form a maximum complex size of n strands. It is of note that this setting did not adequately represent the actual interaction in solution where higher order DNA complexes might form, especially when HCR was involved. However, this simple analysis sufficed in capturing the initial circuit events and background noise formation. The Na⁺ and Mg²⁺ concentrations were set as the same values used in the respective experiments.

### Preparation of Split Proximity Circuit Components

The hybridization chain reaction (HCR) hairpins were prepared just before the experiments. 1 μM of hairpin 1 (HP1) tagged with Cy5 and hairpin 2 (HP2) tagged with Cy3 were heated separately in in reaction buffer to 95° C. for 5 minutes and snap-cooled on ice for at least 15 minutes.

The basic composition of the reaction buffer is 1×PBS (pH 7.4) and can be supplemented with other salts such as 1-10 mM MgCl₂. Other buffers, e.g. Tris, HEPES and saline-sodium citrate, can be used as well. Thereafter, the circuit components were mixed to a typical final reaction concentration of, for example, 20 nM HP-I1, 20 nM 12, 40 nM HP1 and 20 nM HP2. In this case, the ratio of components used is 1:1:2:1 (Structure I:Structure II:Structure III:Structure IV).

### Fluorescence Measurement for HCR-FRET Readout

All reactions were performed at room temperature for a reaction volume of 20 μL in the 384-well black plate.

The Förster resonance energy transfer (FRET) readout was measured on a microplate reader or imaged under a confocal microscope. The z-position and gain was optimized using the software tool for each set of DNA readout concentration and kept constant throughout the analysis.

### SEQUENCES

Table 3 provides a list of the DNA sequences used in the experiments disclosed herein. The sequences were obtained from previous split proximity circuit (SPC) designs, while additional domains were partially designed using NUPACK web server with sequence constraints.

| | | |
|---|---|---|
| SEQ ID | | |
| NO: | Strand | Sequence |

Target / Reporter Strands

| | | |
|---|---|---|
| 1 | ST (split trigger) | GAG TGG ATG GTG AAG GTG AAG GTA |
| 2 | HP1, showing exemplary | GTT GGA ATT GGG AGT AAG GGC |
| | placement of the detection moiety | /iCy5/TGT GAT GCC CTT ACT CCC |
| 3 | HP2, showing exemplary | GCC CTT ACT CCC AAT TCC AAC GGG |
| | placement of the detection moiety | AGT AAG GGC ATC ACA /Cy3/ |
| 4 | F (Fluorophore), showing | GTT GGA ATT GGG AGT AAG GGC /3- |
| | exemplary placement of the | FAM/ |
| | detection moiety | |
| 5 | Q(Quencher), showing | /5IABkFQ/ GCC CTT ACT CCC |
| | exemplary placement of the | |
| | detection moiety | |
| 59 | Exemplary target sequence for | CGGCTGTCCAACCACATCTCCTCC |
| | Single Nucleotide Mismatch | |
| | Detection_(perfectly matched) | |
| 60 | Exemplary target sequence for | CGGCTGTCCAACCACATCTTCTCC |
| | Single Nucleotide Mismatch | |
| | Detection (single mismatched) | |
| 61 | miR-21 | UAGCUUAUCAGACUGAUGUUGA |
| 62 | miR-let-7a | UGAGGUAGUAGGUUGUAUAGUU |
| 63 | miR-9 | UCUUUGGUUAUCUAGCUGUAUGA |
| 64 | miR-29 | ACUGAUUUCUUUUGGUGUUCAG |
| 65 | SARS-CoV-2 (NC_045512.2) | TCTGGTTACTGCCAGTTGAATCTGAGGGT |
| | | CCACCAAACGTAATGCGGGGTGCATTTC |
| | | GCTGATTTTGGGGTC |
| 66 | HP-I1 (with biotin-tag) | Biotin- |
| | | TTTTTTTTTTTTTTTGTGCAGTGTTCAATT |
| | | CCAACGAACAC |
| 67 | I2 (with biotin-tag) | GCCCTTACTCCCACTGCACTTTTTTTTTTT |
| | | TTTT -Biotin |
| 68 | Duplex-cI2 | GTGCCCGGAGTAAGGGC |
| 69 | SM-I1 | TACCTTCACCTTTTTTTTTTTTTTTTTGTG |
| | | CCCTTAATTCCAACAAGG |
| 70 | Xprobe-F | GTTAAATCGTGGATAGTAGACTTCGCAC |
| | | /3Rox_N/ |
| 71 | Xprobe-Q | /5IAbRQ/ |
| | | GTGCGAACAGGTACATTTGCTCGTCCTT |
| 72 | XF-P | AAGGACGAGCAAATGTACCTGGCCCTTA |
| | | CTCC |
| 73 | XQ-PC | GTTGGAATTGGGAGTAAGGGCGTCTACT |
| | | ATCCACGATTTAAC |
| 74 | HP1 (AuNP) | GTTGGAATTGGGAGTAAGGGCTGTGATG |
| | | CCCTTACTCCC |
| 75 | HP2 (AuNP) | GCCCTTACTCCCAATTCCAACGGGAGTA |
| | | AGGGCATCACA |
| 76 | HP1 (DNAzyme) | GTTGGGAAAGTTGGAATTGGGAGTAAGG |
| | | GCTGTGATGCCCTTACTCCCTGGGTAGGG |
| | | CGG |
| 77 | HP2 (DNAzyme) | GTTGGGAAAGCCCTTACTCCCAATTCCA |
| | | ACGGGAGTAAGGGCATCACA |

Hairpin-Modified Association Toehold

| | | |
|---|---|---|
| 6 | I1-HP1 | TAC CTT CAC CTT TTT TTT TTT TTT TTT GTG C GGC A TT C |
| | | AAT TCC AAC TGCC |
| 7 | I2-HP1 | GCC CTT ACT CC TGCC GCA C TTT TTT TTT TTT TTT CAC |
| | | CAT CCA CTC |
| 8 | I1-HP2 | TAC CTT CAC CTT TTT TTT TTT TTT TTT GTG C GGC TT C |
| | | AAT TCC AAC A GCC |
| 9 | I2-HP2 | GCC CTT ACT CC GCC GCA C TTT TTT TTT TTT TTT CAC |
| | | CAT CCA CTC |
| 10 | I1-HP3 | TAC CTT CAC CTT TTT TTT TTT TTT TTT GTG C CC TT C |
| | (A4-6) | AAT TCC AAC AA GG |
| 11 | I2-HP3 | GCC CTT ACT CC GG GCA C TTT TTT TTT TTT TTT CAC CAT |
| | (A4-6) | CCA CTC |
| 12 | I1-HP4 | TAC CTT CAC CTT TTT TTT TTT TTT TTT GTG C GCC TT C |
| | | AAT TCC AAC AA GGC |
| 13 | I2-HP4 | GCC CTT ACT CC GGC GCA C TTT TTT TTT TTT TTT CAC |
| | | CAT CCA CTC |
| 14 | I1-HP5 | TAC CTT CAC CTT TTT TTT TTT TTT TTT GTG CA GGC TT C |
| | | AAT TCC AAC AA GCC |
| 15 | I2-HP5 and | GCC CTT ACT CC GCC TGC AC TTT TTT TTT TTT TTT CAC |
| | I2-HP6 | CAT CCA CTC |
| 16 | I1-HP6 | TAC CTT CAC CTT TTT TTT TTT TTT TTT GTG CA GGC TTC |
| | | AAT TCC AAC GAA GCC |
| 17 | I1-HP7 | TAC CTT CAC CTT TTT TTT TTT TTT TTT GTG CA GTG TTC |
| | (A5-8) | AAT TCC AAC GAA CAC |
| 18 | I2-HP7 | GCC CTT ACT CC CAC TGC AC TTT TTT TTT TTT TTT CAC |
| | (A5-8) and | CAT CCA CTC |
| | I2-HP8 | |
| 19 | I1-HP8 | TAC CTT CAC CTT TTT TTT TTT TTT TTT GTG CA GTG TTC A |
| | | ATT CCA AC TGA A CAC |
| 20 | I1-HP9 | TAC CTT CAC CTT TTT TTT TTT TTT TTT AGT AGT GGT TTC |
| | (A6-9) | AAT TCC AAC GAA ACC |
| 21 | I2-HP9 | GCC CTT ACT CC ACC ACT ACT TTT TTT TTT TTT TTT CAC |
| | (A6-9) | CAT CCA CTC |
| 46 | HP-I1 (7) | CCTCATCCCTACTTTTTTTTTTTTTTTGCTAGACGAATTTCAA |
| | | TTCCAACTGAAATTCG |
| 47 | I2(7) | GCCCTTACTCCATTCGTCTAGCTTTTTTTTTTTTTTTATCCATC |
| | | TCCAC |
| 48 | HP-I1 (8) | TACCTTCACCTTTTTTTTTTTTTTTGCTAGACGAATTTCAATT |
| | and HP-I1 | CCAACTTGAAATTCG |
| | (9) | |
| 49 | I2 (8) | GCCCTTACTCCATTCGTCTAGCAATTTTTTTTTTTTTTTCACC |
| | | ATCCACTC |
| 50 | I2 (9) | GCCCTTACTCCATTCGTCTAGCTTTTTTTTTTTTTTTCACCAT |
| | | CCACTC |
| 51 | HP-I1 (10) | TACCTTCACCTTTTTTTTTTTTTTTGCTAGACGAATTTTCAAT |
| | | TCCAACTTGAAAATTCG |
| 52 | I2 (10) | GCCCTTACTCCAATTCGTCTAGCTTTTTTTTTTTTTTTCACCA |
| | | TCCACTC |
| 53 | HP-I1 (11) | TAC CTT CAC CTT TTTTTTTTTTTTT GCTAGA CGAATT |
| | | TTCAA TTCCAAC TTGAA AATTCG |
| 54 | I2 (11) | GCC CTT ACT CC AATTCG TCTAGCTTTTTTTTTTTTTTT |
| | | CAC CAT CCA CTC |
| 55 | HP-I1 (12) | TACCTTCACCTTTTTTTTTTTTTTTGCTATTCGAATTTTCAATT |
| | | CCAACTTGAAAATTCG |
| 56 | I2 (12) | GCCCTTACTCCAATTCGAATAGCTTTTTTTTTTTTTTTCACCA |
| | | TCCACTC |
| 57 | HP-I1 (13) | TACCTTCACCTTTTTTTTACCCGATTCAATTCCAACAATCG |
| 58 | I2 (13) | GCCCTTACTCCTCGGGTTTTTTTCACCATCCACTC |

Association Toehold (Conventional Design)

| | | |
|---|---|---|
| 22 | I1-A4 | TAC CTT CAC CTT TTT TTT TTT TTT TTT GTG C TT C AAT TCC |
| | | AAC |
| 23 | I2-A4 | GCC CTT ACT CC GCA C TTT TTT TTT TTT TTT CAC CAT CCA |
| | | CTC |
| 24 | I1-A5 | TAC CTT CAC CTT TTT TTT TTT TTT TTT GTG CA TT C AAT TCC |
| | | AAC |
| 25 | I2-A5 | GCC CTT ACT CC TGC AC TTT TTT TTT TTT TTT CAC CAT CCA |
| | | CTC |
| 26 | I1-A6 | TAC CTT CAC CTT TTT TTT TTT TTT TTT AGT AGT TT C AAT |
| | | TCC AAC |
| 27 | I2-A6 | GCC CTT ACT CC ACT ACT TTT TTT TTT TTT TTT CAC CAT CCA |
| | | CTC |

Sequences Used in Example Illustrations

| | | |
|---|---|---|
| 28 | PM-I1 and | GAG GAG ATG TTT TTT TTT TTT TTT AGT AGT GGT TTC |
| | I1-HP9 (A6- | AAT TCC AAC GAA ACC |
| | 9) | |
| | (for single | |
| | nucleotide | |
| | mismatch | |
| | detection) | |
| 29 | PM-I2 and | GCC CTT ACT CCA CCA CTA CTT TTT TTT TTT TTT TTG |
| | I2-HP9 (A6- | TTG GAC AGC CG |
| | 9) | |
| | (for single | |
| | nucleotide | |
| | mismatch | |
| | detection) | |
| 30 | miR-21 (HP- | TCA ACA TCA GTT TTT TTG TGC AGT GTT CAA TTC CAA |
| | I1) | CGA ACA C |
| 31 | miR-21 (I2) | GCC CTT ACT CCC ACT GCA CTT TTT TTC TGA TAA GCT A |
| 32 | miR-let-7a | AAC TAT ACA AC TTT TTT TTT TTT TTT GTG C TT C AAT |
| | (HP-I1) | TCC AAC |
| 33 | miR-let-7a | GCC CTT ACT CCG CAC TTT TTT TTT TTT TTT TAC TAC |
| | (12) | CTC A |
| 34 | miR-9 (HP- | TCA TAC AGC TAT TTT TTT TTT TTT TTG TGC CCT TCA |
| | I1) | ATT CCA ACA AGG |
| 35 | miR-9 (I2) | GCC CTT ACT CCG GGC ACT TTT TTT TTT TTT TTG ATA |
| | | ACC AAA GA |
| 36 | miR-29 (HP- | CGG AAC ACC ATT TTT TTT TTT TTT TGT GCC CTT CAA |
| | I1) | TTC CAA CAA G |
| 37 | miR-29 (12) | GCC CTT ACT CCG GGC ACT TTT TTA AAG AAA TCA GT |
| 38 | SARS-CoV-2 | TGC ACC CCG CAT TAC TTT TTT TTT TTT TTT GTGC CC TT |
| | (HP-I1) | CAATTCCAAC AAGG |
| 39 | SARS-CoV-2 | GCC CTT ACT CCG GGC ACT TTT TTT TTT TTT TTG TTT |
| | (I2) | GGT GGA CCC TC |
| 40 | Thrombin | AGT CCG TGG TAG GGC AGG TTG GGG TGA CTT TTT TTT |
| | (HP-I1) | TTT TTT TTG TGC AGT GTT CAA TTC CAA CGA ACA C |
| 41 | Thrombin | GCC CTT ACT CCC ACT GCA CTT TTT TTT TTT TTT TGG |
| | (I2) | TTG GTG TGG TTG G |
| 42 | I1-HP3 (A4- | SH-TTT TTT TTT TTT TTT TTT TTT TTT TTT TTT GTG CCC |
| | 6) | TTC AAT TCC AAC AAG G |
| | (for protein | (thiolated end “SH-” was later conjugated to antibody) |
| | detection) | |
| 43 | I2-HP3 (A4- | GCC CTT ACT CCG GGC ACT TTT TTT TTT TTT TTT TTT |
| | 6) | TTT TTT TTT TT -SH |
| | (for protein | (thiolated end “SH-” was later conjugated to antibody) |
| | detection) | |
| 44 | I1-HP7 (A5- | SH- TTT TTT TTT TTT TTT GTG CAG TGT TCA ATT CCA |
| | 8) | ACG AAC AC |
| | (for protein | (thiolated end “SH-” was later conjugated to antibody) |
| | detection) | |
| 45 | I2-HP7 (A5- | GCC CTT ACT CCC ACT GCA CTT TTT TTT TTT TTT T -SH |
| | 8) | (thiolated end “SH-” was later conjugated to antibody) |
| | (for protein | |
| | detection) | |

1. A composition for detecting the presence of one or more target(s) of interest, wherein the composition comprises the following components:
a first hairpin initiator molecule according to structure I: 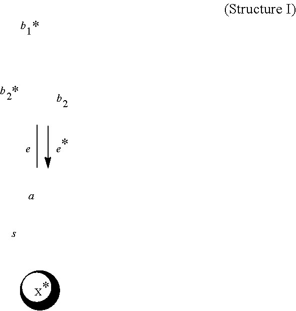
wherein the structure comprises domains a, b₁*, b₂, b₂*, e, e*, s and x*;
wherein neighbouring domains are connected directly to each other or via a linker;
wherein domain x* binds, or is complementary, to the one or more target(s) of interest;
wherein domain b₁* forms a hairpin loop;
wherein domains b₂* and b₂ are of the same length and are complementary to each other;
wherein domains b₂* and b₂ are at least 2 nucleotides in length;
wherein domains e and e* are of the same length, are at least 2 nucleotides in length, and are complementary to each other;
wherein domain a is at least 3 nucleotides in length;
wherein domain s is a spacer; and
a second initiator molecule according to structure II: 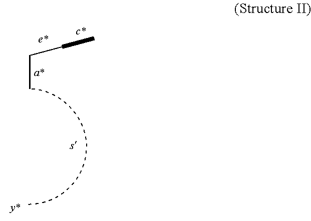
wherein the structure comprises domains a*, c*, e*, s′ and y*;
wherein domain y* is complementary, or binds to, the one or more target(s) of interest;
wherein domains a and a* are complementary to each other;
wherein domain e* of structure II is at least 2 nucleotides in length and binds to domain e of structure I upon binding the target of interest,
wherein domain c* is capable of binding to a signal generating molecule/signal generating complex; and
wherein domain s′ is a spacer,
wherein the length of domains s and s′ are selected to allow domains b₁*, b₂* and c* to be adjacent to each other upon binding.

2. The composition of claim 1, wherein all domains, except domains x*, y*, s, and s′, are nucleic acid sequences.

3. The composition of claim 1, wherein domains x* and y* are selected from the group consisting of nucleic acid sequence, protein sequence, including post-translational modified versions thereof, antibody, antigen, and small molecule.

4. The composition of claim 1, wherein domain a* is between 3 to 10 nucleotides in length; and/or wherein domain b₁* is at least 3 nucleotides long; and/or wherein domain b₂* is between 2 to 6 nucleotides long; and/or wherein domain c* is at least the length of domain b₁*; and/or wherein domain e* is between 2 and 8 nucleotides long; and/or wherein domain s is between 1 to 50 nm long.

5. (canceled)

6. (canceled)

7. (canceled)

8. (canceled)

9. (canceled)

10. The composition of claim 1, wherein domain a* is between 3 to 10 nucleotides in length; wherein domain b₁* is at least 3 nucleotides long; wherein domain b₂* is between 2 to 6 nucleotides long; wherein domain c* is at least the length of domains b₁*; and wherein domain e* is between 2 and 8 nucleotides long.

11. The composition of claim 1, wherein domain a* is between 3 to 10 nucleotides in length; wherein domain b₁* is at least 3 nucleotides long; wherein domain b₂* is between 2 to 6 nucleotides long; wherein domain c* is at least the length of domains b₁*; and wherein domain e* is between 2 and 8 nucleotides long; and wherein domain s is between 1-50 nm long.

12. (canceled)

13. The composition of claim 1, wherein the one or more target(s) of interest are selected from the group consisting of DNA, RNA, single nucleotide polymorphism (SNP), microRNA (miRNA), genomic DNA, viral DNA, protein, post-translational modified proteins, cell surface receptors, metabolites, lipids, carbohydrates and small molecules.

14. The composition of claim 1, wherein the composition further comprises one or more signal generating molecules/signal generating complexes.

15. The composition of claim 1, further comprising a pair of nucleic acid hairpins comprising a first hairpin reporter (HP1) and a second hairpin reporter (HP2) according to structures III and IV, respectively: 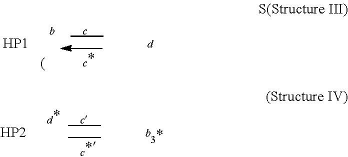
wherein the first hairpin reporter comprises domains b, c, d and c*,
wherein domain b is a 5′ nucleic acid overhang with a length between 6 to 12 nucleotides,
wherein domain d is a hairpin loop with a length between 6 to 12 nucleotides,
wherein domain c* is as defined in any one of claims 1 to 15.
wherein the second hairpin reporter (HP2) comprises domains d*, c′, b₃* and c*′,
wherein domain b₃* is a hairpin loop of the same length as domain b,
wherein domain d* is a nucleic acid overhang of the same length as domain d,
wherein domains c′ and c*′ are of the same length, and are capable of binding to each other, and
wherein domains b₃* and d each form a hairpin loop.

16. The composition of claim 15, wherein domain d and d* are complementary to each other; and/or wherein the domains c′ and c*′ are complementary to each other; and/or wherein domain d* is between 6 to 12 nucleotides long.

17. (canceled)

18. The composition of claim 15, wherein the first hairpin reporter (HP1) comprises a first detection moiety; and/or wherein the second hairpin comprises a second detection moiety.

19. (canceled)

20. The composition of claim 18, wherein the detection moiety is selected from the group consisting of fluorophores, small molecules, proteins and inorganic nanomaterials.

21. (canceled)

22. (canceled)

23. A method for detecting the presence of one or more target(s) of interest in a sample, the method comprising:
adding one or more compositions according to claim 1 to the sample;
allowing binding of the one or more compositions to the one or more target(s) of interest thought to be comprised in the sample;
measuring one or more signals resulting from the binding of the composition(s) to the target(s) of interest;
wherein the generation of one or more signals detects the presence of one or more of the target(s) of interest in the sample.

24. The method according to claim 23, wherein each component of the one or more compositions, as defined in claim 1, is added simultaneously or sequentially.

25. The method of claim 23, wherein the first hairpin initiator molecule and the second initiator molecule, as defined in claim 1, bind to at least one target(s) of interest.

26. (canceled)

27. The method of claim 23, wherein the method is performed at a single temperature, at which the composition(s) bound to the target(s) of interest are stable.

28. The method of claim 23, wherein the method is performed in a single reaction vessel.

29. The method according to claim 23, wherein the one or more target(s) of interest are selected from the group consisting of DNA, RNA, single nucleotide polymorphism (SNP), microRNA (miRNA), genomic DNA, viral DNA, protein, protein interactions, post-translationally modified proteins, cell surface receptors metabolites, lipids, carbohydrates and small molecules.

30. The method of claim 23, wherein presence of the one or more target(s) of interest indicates the presence of a disease.

31. A method of identifying a disease, the method comprising
adding one or more compositions according to claim 1 to a sample obtained from a subject suspected to have the disease;
allowing binding of the one or more compositions to the one or more target(s) of interest;
measuring one or more signals resulting from the binding of the composition(s) to the target(s) of interest; and
identifying the disease, wherein the presence of one or more signals indicates the presence of one or more of the target(s) of interest in the sample; and wherein the one or more of the target(s) of interest are disease-specific.

32. (canceled)

33. (canceled)

34. (canceled)

35. (canceled)

36. (canceled)
